(19) United States
(12) Patent Application Publication
(10) Pub. No.: US 2022/0330832 A1
(43) Pub. Date: Oct. 20, 2022

(54) Modulating Photosensitive Proteins with Mechanoluminescent Particles

(71) Applicant: The Board of Trustees of the Leland Stanford Junior University, Stanford, CA (US)

(72) Inventors: Fan Yang, Redwood City, CA (US); Zihao Ou, Redwood City, CA (US); Guosong Hong, Cambridge, MA (US); Xiang Wu, Redwood City, CA (US); Paul Chong, Palo Alto, CA (US); Huiliang Wang, Redwood City, CA (US); Xingjun Zhu, Shanghai (CN)

(21) Appl. No.: 17/642,648

(22) PCT Filed: Nov. 25, 2020
(86) PCT No.: PCT/US2020/062376

§ 371 Date: Mar. 11, 2022

Related U.S. Application Data
(60) Provisional application No. 62/941,234, filed on Nov. 27, 2019.

(51) Int. Cl. A61B 5/00; A61K 41/00; A61K 49/00; A61N 5/06

(52) U.S. Cl. CPC A61B 5/0097; A61K 41/0057; A61K 49/0093; A61N 5/062

(57) ABSTRACT

Provided are methods of contacting a tissue inside a subject with light by applying an ultrasound signal to a photoexcited mechanoluminescent particle while the mechanoluminescent particle is inside the subject and in proximity to the tissue, thereby causing the mechanoluminescent particle to emit light that contacts the tissue. Provided are systems and kits for performing such methods.

### CROSS-REFERENCE TO RELATED APPLICATIONS

This application claims priority to U.S. Provisional Application No. 62/941,234, filed on Nov. 27, 2019, the disclosure of which is incorporated herein by reference in its entirety.

### GOVERNMENT RIGHTS

This invention was made with government support under grant number AG056636 awarded by the National Institutes of Health, grant number MH117490 awarded by the National Institutes of Health, and grant number DGE-1656518 awarded by the National Science Foundation. The government has certain rights in the invention.

### INTRODUCTION

Contacting a tissue inside a subject with light can be used in several applications, including optogenetics, photodynamic therapy, selective gene editing, and fluorescent imaging. In optogenetics, a neuron that includes a photosensitive protein can be contacted with light, thereby modulating the neuron. Thus, optogenetics allows for the study of neural circuits by specifically activating or deactivating particular neurons. In photodynamic therapy, a photosensitive compound is administered to a subject. Upon being contacted by light, the photosensitive compound catalyzes the conversion of oxygen into reactive oxygen species, which damage and kill nearby cells such as cancer cells. In fluorescent imaging, a fluorescent compound is administered to a subject and then contacted with light, causing it to emit fluorescent light. The fluorescent light can be measured in order to image the tissue of the subject.

However, for the wavelengths of light commonly used in these applications, bodily tissues have high absorption coefficients. As such, light applied to an external surface of a subject can only penetrate a short distance into a subject before being absorbed. Thus, most tissues in a subject cannot be sufficiently exposed to external light of such wavelengths in order to allow for practical use in optogenetics, photodynamic therapy, or fluorescent imaging. Implanting fiber optic wires in order to deliver light, such as in optogenetics, involves various disadvantages and complications.

### SUMMARY

Provided are methods of contacting a tissue inside a subject with light by applying an ultrasound signal to a photoexcited mechanoluminescent particle while the mechanoluminescent particle is inside the subject and in proximity to the tissue, thereby causing the mechanoluminescent particle to emit light that contacts the tissue. In some of such methods, the tissue comprises a neuron that comprises a photosensitive protein, and wherein the emitted light modulates the photosensitive protein. Some of such methods include imaging the tissue by measuring light emitted by the mechanoluminescent particle. In some of such methods, the emitted light contacts a photosensitizer, thereby causing generation of a reactive oxygen species. Provided are systems and kits for performing such methods.

### BRIEF DESCRIPTION OF THE DRAWINGS

FIG. 1A shows ZnS:Ag, Co/ZnS nanoparticles act as rechargeable light sources in blood circulation for sono-optogenetics. (A) Schematic showing the sono-optogenetic neural modulation via ultrasound-triggered light emission from ZnS:Ag, Co/ZnS nanoparticles circulating in the blood circulation system

FIG. 1B shows mechanism of ultrasound-triggered light emission from ZnS:Ag, Co/ZnS nanoparticles. In this drawing, the electron trap is created in the host material of ZnS by Co²⁺ dopant ions, causing the photoexcited electrons to be trapped after absorption of 400 nm excitation light. FUS allows the trapped electrons to transfer energy into the luminescent centers created by Ag⁺ dopant ions, resulting in 470-nm light emission.

FIG. 1C shows schematic showing blood circulation of ZnS:Ag, Co/ZnS nanoparticles, transporting the 400-nm photoexcitation energy at superficial vessels into 470-nm emission in deep-brain regions for optogenetic stimulation

FIG. 1D shows representative high-resolution TEM image of ZnS:Ag, Co/ZnS nanoparticles.

FIG. 1E shows distribution of the hydrodynamic diameters of ZnS:Ag, Co/ZnS nanoparticles in phosphate buffered saline (PBS, blue) and Dulbecco's Modified Eagle Medium (DMEM) supplemented with 10% fetal bovine serum (FBS, red), both revealed by DLS measurements.

FIG. 2A shows detailed mechanism of ultrasound-triggered light emission from ZnS:Ag, Co/ZnS nanoparticles. (A) ZnS:Ag, Co/ZnS nanoparticles absorb the 400-nm light to excite an electron in the valence band to the conduction band.

FIG. 2B shows the excited electron is trapped in the defect state create by the Co²⁺ dopant ion.

FIG. 2C shows focused ultrasound (FUS) creates mechanical stress and charge separation in the piezoelectric ZnS matrix, effectively tilting the conduction band to equilibrate with the electron trap, thus detrapping the electron back to the conduction band.

FIG. 2D shows the ‘detrapped’ electrons transfer energy to the Ag⁺ dopant ions.

FIG. 2E shows the Ag⁺ dopant ions act as emission centers to release the transferred energy in 470-nm light.

FIG. 3 shows UV-Vis-NIR spectrum of the ZnS:Ag, Co/ZnS nanoparticle suspension.

FIG. 4 shows XRD spectrum of the ZnS:Ag, Co/ZnS nanoparticles and the standard peaks reported in the Joint Committee on Powder Diffraction Standards (JCPDS) card no. 36-1450 for wurtzite ZnS.

FIG. 5, panel A, shows luminescence properties of ZnS:Ag, Co/ZnS nanoparticles. (A) Photoluminescence spectra of undoped ZnS (cyan), ZnS:Ag, Co (gray) and ZnS:Ag, Co/ZnS nanoparticles (blue) under 365-nm photoexcitation.

FIG. 5, panel B, shows mechanoluminescence spectra of undoped ZnS (cyan), ZnS:Ag, Co (gray), and ZnS:Ag, Co/ZnS nanoparticles (blue) under FUS excitation.

FIG. 5, panel C, shows normalized photoluminescence (green) and mechanoluminescence (blue) spectra of ZnS:Ag, Co/ZnS nanoparticles, overlaid with the absorption spectrum of ChR2 (black dashed curve with pink fill).

FIG. 5, panel D, shows schematic of the artificial circulatory system, where the 400-nm LED provides photoexcitation to charge the circulating ZnS:Ag, Co/ZnS nanoparticles, and the FUS transducer triggers the release of stored energy into 470-nm light mission in the PDMS phantom.

FIG. 5, panel E, shows intensity of 470-nm emission from ZnS:Ag, Co/ZnS nanoparticles in the artificial circulatory system under repetitive FUS stimulation (red ticks) and continuous 400-nm recharging light (violet bar).

FIG. 5, panel F, shows intensity of 470-nm emission from ZnS:Ag, Co/ZnS nanoparticles in the artificial circulatory system under repetitive FUS stimulation (red ticks) and discontinued 400-nm recharging light (violet bar).

FIG. 6 shows photographs of a PDMS phantom placed at the focus of the ultrasound transducer, exhibiting minimal emission, visible blue emission and disappearance of the blue emission under ambient lighting before (a), during (b) and after (c) FUS stimulation. Arrow 1 indicates the FUS transducer and arrow 2 indicates the PDMS phantom with embedded ZnS:Ag, Co/ZnS nanoparticles.

FIG. 7 shows time-resolved luminescence measurements of the PDMS phantom comprising ZnS:Ag, Co/ZnS nanoparticles, with (red) and without (black) FUS excitation after irradiation with 400-nm to recharge the nanoparticles (t=0 s). An exponential decay, which indicates the afterglow of ZnS:Ag, Co/ZnS nanoparticles, is followed by an increase of luminescence intensity by ˜18 fold upon FUS excitation at t=4.5 s (spike in the red curve, indicated by the blue arrow).

FIG. 8 shows schematic of the artificial circulatory system for repetitive emission of 470-nm light triggered by FUS and measured by fiber-coupled PMT,

FIG. 9 shows statistics of latency times between FUS excitation and 470-nm light emission measured from ZnS:Ag, Co/ZnS nanoparticles in an artificial circulatory system. A total of 20 trials are shown in red diamonds, with the height and error bar of bar chart indicating the mean and standard deviation of measurements, respectively.

FIG. 10 shows sono-optogenetic stimulation of spiking HEK cells in vitro. (A) Schematics showing ultrasound-triggered opening of ChR2 channels via conversion to 470-nm light emission by ZnS:Ag, Co/ZnS nanoparticles. (B) Representative extracellular recording traces of cultured spiking HEK cells under different conditions of ChR2 transfection, ZnS:Ag, Co/ZnS nanoparticle presence and FUS stimulation. Timed FUS pulses successfully triggered action potentials of spiking HEK cells only when the cells expressed ChR2 and the artificial circulatory system contained ZnS:Ag, Co/ZnS nanoparticles in the circulation. (C) Overlaid extracellular single-unit spikes recorded from spiking HEK cells expressing ChR2 and sono-optogenetically stimulated by ZnS:Ag, Co/ZnS nanoparticles and FUS. (D) Bar chart summarizing FUS-triggered action potential amplitudes for different groups shown in C with FUS on (red bars) and off (blue bars) from N=82 stimuli per group. ****, p<0.0001. The error bars represent ±1 standard deviation (SD).

FIG. 11 shows sono-optogenetic stimulation of motor activity in vivo. (A) Schematic of in vivo sono-optogenetic stimulation. (B) Photograph of in vivo sono-optogenetic stimulation setup, showing intact scalp and skull of the mouse. (C) Measured equivalent power density of 470-nm emission in local brain tissue by circulating ZnS:Ag, Co/ZnS nanoparticles under repetitive FUS stimulation (red ticks) and continuous 400-nm recharging light (violet bar). (D & E) Photographs of a Thy1-ChR2-YFP mouse (D) and a wild-type (WT) mouse (E) during sono-optogenetic stimulation through intact scalp and skull, before (left) and after (right) injection of ZnS:Ag, Co/ZnS nanoparticles. Red and black lines indicate the kinematics of left and right hindlimbs, respectively. (F & G) Hindlimb kinematics of corresponding Thy1-ChR2-YFP mouse (F) and WT mouse (G) during sono-optogenetic stimulation, with N=4 trials shown for each graph. For each trial, both the starting position and maximum range of motion are shown for each hindlimb, resulting in 8 kinematic diagrams, which are either overlapping or separate depending on the effect of simulation. Contralateral limb activation in ChR2 mouse by sono-optogenetic stimulation is highlighted in red kinematic diagrams. (H) Representative hindlimb displacement over repetitive FUS pulses from a Thy1-ChR2-YFP mouse injected with ZnS:Ag, Co/ZnS nanoparticles. (I) Statistics of left hindlimb displacement in different groups of subjects (n=3 per group) in response to FUS excitation. The bar heights indicate the mean, and the error bars indicate standard error of the mean (SEM). ****, p<0.0001; N.S., not significant.

FIG. 12 shows a representative plot of the concentration of the ZnS:Ag, Co/ZnS nanoparticles in the blood versus time after tail-vein injection, as determined by the photoluminescence of the blood samples. A first-order exponential fits the data points with a half-life of circulation for the nanoparticles of 127.8±45.3 min.

FIG. 13 shows peak pressure measured by the hydrophone as a function of percent amplitude for the FUS system. Linear fitting results in the empirical formula of P=0.101 A+0.178, where P is pressure in MPa, and A is amplitude in %.

FIG. 14 shows in vivo temperature at the ultrasound focus of the mouse brain with the following protocol: FUS frequency: 1.5 MHz; repetition frequency: 1 Hz; duty cycle: 10%; spatial peak pulsed average intensity: 10.0 W/cm². Ultrasound was applied after t=0 s and the duration was indicated with the red shade.

FIG. 15 shows statistics of left and right hindlimb displacement in different groups of subjects (n=3 per group) during FUS excitation. The bar heights indicate the mean, and the error bars indicate standard error of the mean (SEM). ****, p<0.0001; N.S., not significant.

FIG. 16 shows ZnS:Ag, Co nanoparticles without mechanoluminescence enhancement by additional coating process do not elicit hindlimb motion under the same sono-optogenetic stimulation protocol (FUS frequency: 1.5 MHz; repetition frequency: 1 Hz; duty cycle: 10%; spatial peak pulsed average intensity: 10.0 W/cm²). (A) Photograph of a Thy1-ChR2-YFP mouse during sono-optogenetic stimulation through intact scalp and skull after injection of ZnS:Ag, Co nanoparticles without the additional coating process. Red and black lines indicate the kinematics of left and right hindlimbs, respectively. (B) Hindlimb kinematics of the mouse shown in (A) during sono-optogenetic stimulation over N=4 trials. For each trial, both the starting position and maximum range of motion are shown for each hindlimb, resulting in 8 kinematic diagrams for each limb. (C) Statistics of left hindlimb displacement from a group of n=3 Thy1-ChR2-YFP mouse injected with ZnS:Ag, Co nanoparticles with (red) and without (blue) FUS excitation. The bar heights indicate the mean, and the error bars indicate standard deviation (SD). N.S., not significant.

FIG. 17 shows comparison of different methods for in vivo optogenetic stimulation.

FIG. 18 shows H&E staining of major organs from mice at 3 and 20 days post-intravenous administration of ZnS:Ag, Co/ZnS nanoparticles (middle and bottom rows, respectively) or blank PBS (top row). No noticeable tissue damage or pathological lesion was found in organs from either ZnS:Ag, Co/ZnS nanoparticles or PBS treated groups.

FIG. 19 shows a transmission electron microscopy (TEM) image of Sr₂MgSi₂O₇:Eu²⁺, Dy³⁺ nanoparticles.

FIG. 20 shows an X-ray diffraction (XRD) pattern of Sr₂MgSi₂O₇:Eu²⁺, Dy³⁺ nanoparticles and standard JCPDS card: 75-1736.

FIG. 21 shows the light emission intensity from the PDMS phantoms with the same concentration of Sr₂MgSi₂O₇:Eu²⁺, Dy³⁺ or ZnS:Ag, Co/ZnS nanoparticles. A 1-s FUS pulse was given at t=7.5 s to trigger mechanoluminescence. Note that the curve for ZnS:Ag, Co/ZnS is multiplied by a factor of 50 for easy comparison.

### DETAILED DESCRIPTION

Provided are methods of contacting a tissue of a subject with light by applying an ultrasound signal to a photoexcited mechanoluminescent particle while the mechanoluminescent particle is in proximity to the tissue of the subject, thereby causing the mechanoluminescent particle to emit light that contacts the tissue. In some of such methods, the tissue comprises a neuron that comprises a photosensitive protein, and wherein the emitted light modulates the photosensitive protein. Some of such methods include imaging the tissue by measuring light emitted by the mechanoluminescent particle. In some of such methods, the emitted light contacts a photosensitizer, thereby causing generation of a reactive oxygen species. Provided are systems and kits for performing such methods.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth.

It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The terms “applying” and “administering” are used interchangeably to refer to causing a subject to receive a treatment. For example, applying an ultrasound signal means generating an ultrasound signal and directing it into a region of the subject's body. As another example, administering a particle to the subject means moving the particle inside the body of the subject.

The terms active agent, active pharmaceutical ingredient, pharmacologically active agent, and drug are used interchangeably herein to refer to a chemical material or compound which, when administered to an organism (human or animal) induces a desired pharmacologic and/or physiologic effect by local and/or systemic action.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in viral titers).

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of a compositions is an amount that is sufficient, when administered to the individual, to palliate, ameliorate, stabilize, reverse, prevent, slow or delay the progression of a disease state (e.g., cancer, etc.) present in the subject.

As used herein, the terms “determining,” “measuring,” “assessing,” and “assaying” are used interchangeably and include both quantitative and qualitative determinations.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

### Methods

As described above, provided are methods of contacting a tissue of a subject with light by applying an ultrasound signal to a photoexcited mechanoluminescent particle while the mechanoluminescent particle is in proximity to the tissue of the subject, thereby causing the mechanoluminescent particle to emit light that contacts the tissue.

As used herein, the term “particle” refers an object in a solid state of matter. In some cases, the particle is small enough that it can travel through a blood vessel of the subject. For example, some blood vessels in humans have an inner diameter ranging from 8 μm to 25 mm. In some cases, the particle has a dimension ranging from 1 nm to 1 μm, i.e. the particle is a nanoparticle. The dimension can be a length, width, height, or diameter.

The term “mechanoluminescent” means that the particle can luminesce in response to a mechanical force, i.e. the particle can emit light in response to a mechanical force. The mechanoluminescent emission of light can include the emission of a single photon of light, or of two or more photons of light, such as 5 photons or more or 100 photons or more. In other words, when a single mechanoluminescent particle is acted upon by an appropriate mechanical force, it can emit 1 or more photons of light, such as 2 photons or more, 5 photons or more, or 100 photons or more. The mechanoluminescent emission of light can also include the emission of light of an intensity that is detectable by a light detector.

Before a mechanical force causes the particle to luminesce, the particle is first photoexcited. “Photoexcitation” means that the particle absorbs one or more photons of light such that the particle is excited into a higher energy state. In some cases, the particle is in an excited electronic energy state, i.e. it is not in the ground electronic energy state. The terms “state” and “level” are used interchangeably herein. For example, in some cases the particle is a semiconductor or an insulator, i.e. the particle has a band gap between the valence band and the conduction band where few or no electronic states exist. In such cases, an absorbed photon can excite an electron from the valence band to the conduction band. In some cases, the particle has a bandgap energy ranging from 2 eV to 5 eV, such as from 3 eV to 4 eV. In other cases, the excitation energy from an absorbed photon is localized to a few atoms, such as 20 atoms or less, and the excitation energy does not affect population of a valence band or conduction band. Since photons can excite the particle in different ways, the “photoexcited mechanoluminescent particle” can also be referred to as the “excited mechanoluminescent particle”.

The photoexcitation of the mechanoluminescent particles can be either active or passive. For instance, in active photoexcitation a light source is activated for the purpose of photoexciting the particles. In some cases, the photoexcitation light source emits a photoexcitation photon that has a wavelength ranging from 250 nm to 650 nm, such as from 300 nm to 550 nm or from 350 nm to 450 nm. In some cases, 80% or more of the photons emitted by the light source are within such wavelength ranges, such as 90% or more or 95% or more. The wavelength of light can be varied in order to achieve the most efficient photoexcitation of the particle, e.g. by choosing a wavelength that corresponds to a high absorption coefficient for the particle, such as one that corresponds to a band gap of the particle, but a wavelength that has a low absorption coefficient for tissue, such that the photon can penetrate deep into the tissue with a minimal chance of being absorbed by the tissue. The excitation light source can be, for example, a laser. In some instances, the active photoexcitation is performed by directing light onto the skin or other outer surface of the subject, e.g. so that mechanoluminescent particles traveling through a blood vessel near the surface of the skin are photoexcited. For example, the blood vessel can be 20 mm or less below the surface of the skin, such as 10 mm or less, 5 mm or less, 2 mm or less, or 1 mm or less. Since the skin and other body tissues will absorb photoexcitation light, it is preferable to select a blood vessel that is relatively close to the skin, since this will allow a greater fraction of the photons from the light source to reach the particles and photoexcite the particles. In addition, since the photoexcited particles can spontaneously decay to a non-excited energy state after a period of time, sometimes the method involves photoexciting the particles near the location where the ultrasound will be applied. For example, if the method involves treating a particular brain region, a blood vessel in the neck or head can be selected that carries blood to the particular brain region. Applying light to the skin covering the selected blood vessel can photoexcite the particles, which will then travel directly to the particular brain region, where they can be subjected to ultrasound that causes them to mechanoluminesce. As such, the particle can also be referred to as an “ultrasound-luminescent particle”. In some cases, the distance between the photoexcitation of the particles and the mechanoluminesce of the particles is 100 cm or less, such as 50 cm or less, 25 cm or less, or 10 cm or less. In other cases, the particles are actively photoexcited before being moved inside the subject, e.g. before being intravenously injected into the subject. In some cases, the time between the photoexcitation and the luminescence of the particle is 15 minutes or less, such as 5 minutes or less, or 1 minute or less.

In passive photoexcitation, the particles are photoexcited by light from a light source that was already activated for a different purpose, e.g. the particles might be photoexcited by ceiling lights in an operating room that also provide light for medical professionals to see.

After the mechanoluminescent particles are photoexcited, an ultrasound signal is applied to the particles, causing them to emit light. The terms “ultrasound signal” and “ultrasound” are used interchangeably to refer to sound waves with frequencies that are above what a human can hear, e.g. above 20 kHz. Sound waves are physical vibrations that propagate through a medium, e.g. through air, water, bone, and muscle. In some cases the ultrasound has a frequency ranging from 20 kHz to 100 MHz, such as from 100 kHz to 15 MHz, or from 500 kHz to 5 MHz. The ultrasound signal can be a focused ultrasound signal (FUS) or an unfocused ultrasound signal. In focused ultrasound, the ultrasound emitting device is configured such that the highest intensity ultrasound signal is located not only at a particular angle relative to the device, but also at a particular distance away from the ultrasound emitter part of the ultrasound emitting device. For instance, the highest intensity ultrasound signal can be located between 10 mm and 150 mm away from the end of the ultrasound emitter, e.g. 10 mm to 150 mm below the skin, such as between 30 mm and 75 mm. A medical professional can change the settings on the ultrasound emitting device, thereby changing the desired depth of highest ultrasound intensity. This allows for lower intensity ultrasound at a more superficial location, such as the prefrontal cortex of the brain, while also providing for high intensity ultrasound at an interior location, such as Broca's area of the brain, which is located underneath the prefrontal cortex. In contrast, in unfocused ultrasound the highest intensity is at or near the surface of the skin or tissue, with ultrasound intensity decreasing with increasing distance from the emitter.

The ultrasound is applied to the photoexcited particle when the photoexcited particle is in proximity to the tissue of the subject. As used herein, “in proximity to” means that at least one of the photons emitted by the particle will contact the tissue. For example, the tissue can be a group of neuron cells in the auditory complex of the brain, and the particle can be located inside a blood vessel adjacent to the auditory complex. A photon emitted by the particle can pass through the liquid of the blood vessel, pass through the wall of the blood vessel, and then enter a neuron of the auditory complex. In such an embodiment, the photoexcited mechanoluminescent particle was in proximity to the auditory complex tissue since the emitted photon contacted a cell of the target tissue. As described above, photons can be absorbed by body tissue, wherein the degree of absorption depends on the wavelength of light, the type of tissue, and the distance of tissue the photon is passing through. As such, the distance referred to by the term “in proximity to” depends on certain aspects of the method, and is not limited to a particular range. However, in some cases the particle is 10 mm or less from the tissue, such as 1 mm or less, 100 μm or less, or 10 μm or less. The term “in proximity to” also includes cases wherein the particle is within the tissue that is to be contacted with light. For example, the particle can be within a blood vessel inside the auditory complex, or it can be within a blood vessel inside a cancerous tumor of the liver.

In some cases the wavelength of photoexcitation light absorbed by the mechanoluminescent particle is different from the wavelength of light that is emitted by the particle upon being contacted by the ultrasound signal. As described above, the photoexcitation photon can have wavelength ranging from 250 nm to 650 nm, such as from 300 nm to 550 nm or from 350 nm to 450 nm. In some cases, the emitted photon has a wavelength ranging from 350 nm to 2000 nm, such as from 400 nm to 1700 nm, from 400 nm to 1300 nm, from 400 nm to 1000 nm, from 400 nm 800 nm, from 400 nm to 650 nm, or from 450 nm to 500 nm. In some cases, 80% or more of the emitted photons have such wavelengths, such as 90% or more or 95% or more. As used herein, the term “Stokes shift” refers to the difference between the absorption and emission wavelengths, and the Stokes shift can range in some embodiments from 20 nm to 100 nm, such as from 60 nm to 80 nm.

The term “tissue” refers to any region of the subject's body. For example, the tissue can be a brain tissue, e.g. the auditory complex, the prefrontal cortex, or the hippocampus, a muscle tissue, a region of bone, and a liver tissue. In cases wherein the tissue is a brain tissue, the term “brain tissue” includes neurons, glia cells, extracellular fluid, and other components of the brain. In some cases the tissue is a cancerous tumor of any part of the body.

The mechanoluminescent particle can be administered by any suitable means, e.g., suspended in a liquid and injected into a blood vessel.

The amount of time between administering the particles to the subject and the application of light can be any suitable length. In some cases, such a time is 180 minutes or less, 120 minutes or less, 60 minutes or less, 30 minutes or less, 10 minutes or less, of 5 minutes or less. In some embodiments, the amount of time between photoexciting the particle, e.g. photoexciting the particle by applying light to a skin region of the subject while the particles are in a blood vessel below the skin region, and when the particle is contacted with ultrasound ranges from 1 second to 60 minutes, such as from 1 second to 10 minutes, or from 1 second to 1 minute. In some cases, the photoexcitation is stopped before the application of ultrasound is begun. In other cases, the ultrasound is started before the photoexcitation is ended. For instance, multiple particles can be administered to the subject, such as 100 or more, 1,000 or more, 10,000 or more, or 100,000 or more. These particles can disperse in the blood, and therefore they will pass under a particle skin region at different points in time. As such, the photoexcitation light can be maintained for a period of time so that most or all of the particles are photoexcited. However, if some of the photoexcited particles have reached proximity to the tissue before all the particles have been photoexcited, the ultrasound can begin before the photoexcitation has ended.

The emission of light from the photoexcited mechanoluminescent particle can be triggered by any suitable mechanical stimulus, e.g. by ultrasound. In some cases, the ultrasound is focused ultrasound. In some cases, the ultrasound signal has a frequency ranging from 150 kHz to 15 MHz, such as from 300 kHz to 5 MHz, or from 600 kHz to 2 MHz. The ultrasound signal can be repeated, i.e. wherein the signal is emitted for a time and then not emitted for a time, at any suitable repetition. In some cases, the ultrasound signal is repeated at a rate ranging from 0.05 repetitions per second to 20 repetitions per second, such as from 0.2 repetitions per second to 5 repetitions per second, or from 0.5 repetitions per second to 2 repetitions per second.

In some cases, the ultrasound signal has a spatial peak pulsed average intensity (I_{SPPA}) at a target neuron ranging from 1 W/cm² to 100 W/cm², such as from 2 W/cm² to 50 W/cm², or from 5 W/cm² to 15 W/cm². As used herein, W/cm² refers to the units W·cm².

In some cases, the time interval between application of the ultrasound signal and the emission of light from the mechanoluminescent particle is 9 ms or less, such as 7 ms or less, 5 ms or less, or 3 ms or less. The subject can be, for example, a human, a primate, a rat, a mouse, a horse, a dog, or a cat.

In some cases, 80% or more of the photon of the excitation light have a wavelength ranging from 350 nm to 450 nm. In some cases, 80% or more of the photons of the light emitted by the mechanoluminescent particle have a wavelength ranging from 420 nm to 520 nm. In some cases, the photoexcitation light is not within the infrared region, i.e. the photoexcitation light does not have a maximum within the range of 700 nm to 1 mm. The excitation light generally does not have a maximum at 300 nm or less.

In addition, methods described herein do not require the generation of a magnetic field in order to cause the photoexcited mechanoluminescent particle to emit light, or to emit light of a particular wavelength. For example, Truby and Planel describe applying a magnetic field of 20 Tesla to a compound to modify its luminescence (Solid-State Electronics, 1996, 40, 1, doi:10.1016/0038-1101(95)00233-2), whereas the strength of Earth's magnetic field is approximately only 0.000025 to 0.000065 Tesla. As such, in some cases the particles are contacted with the ultrasound when they are in a magnetic field with a strength of 0.0001 Tesla or less.

### Optogenetics

Optogenetics is a method that employs light to modulate tissue, such as neurons, that have been genetically modified to express light-sensitive proteins. In some cases, the neurons are neurons in the brain. Thus, by stimulating these brain neurons with light, brain processes and brain regions can be studied. In some cases, the photosensitive proteins are ion channels located in a cell membrane.

Brain studies with optogenetics traditionally employed, for example, fiber optics implanted into the brain to provide the excitatory light. However, such implantation causes various complications associated with the surgery and use, along with disadvantages such as infection risk and damage to brain tissue resulting from the surgery and implantation.

Any suitable photosensitive protein can be employed, such as channelrhodospin-2 (ChR2), VChR1, iC++, ChRmine, or halorhodopsin (HPHR), e.g. a halorhodopsin from Natronomonas (NpHR). In some cases, the method also includes genetically modifying the neuron to express the photosensitive protein, e.g. with CRISPR gene editing.

The method can result, based on configuration, in the hyperpolarization or depolarization of the target neuron or neurons. Hyperpolarization includes partial hyperpolarization and complete hyperpolarization, whereas depolarization includes partial depolarization and complete depolarization.

### Photodynamic Therapy

As the methods allow for light emission deep within the body, the methods can be described as deep-tissue photodynamic therapy. These methods allow for deeper photodynamic therapy than traditional photodynamic therapy, which uses the excitatory light being contacted on an external surface of a patient. For example, if the photosensitizer is stimulated to generate reactive oxygen species with light of about 400 nm, traditional photodynamic therapy would shine concentrated 400 nm light onto an external surface of the subject. However, since the human body tissues, such as skin and fat, significantly absorb in the 400 nm range, the excitatory light could only reach a shallow depth, thereby only being effective at generating reactive oxygen species near the surface of the skin. For example, traditional photodynamic therapy can be used for skin cancer, as the cancer being contacted with reactive oxygen species (ROS) are near the surface.

In contrast, the methods described herein allow for ROS generation deep within a subject. Ultrasound can penetrate deep within the subject, causing the photoexcited mechanoluminescent particle that are deep within the subject to emit light deep within the subject. For example, the methods can be used to treat cancers that are deep within a subject, such as breast cancer, brain cancer, and liver cancer. The methods can also be used to treat infection by a microorganism, e.g. by a bacteria, parasite, or fungus.

### Fluorescent Imaging

As the methods allow for light emission deep within the body, the methods can be described as deep-tissue photodynamic therapy. These methods, which employ a mechanoluminescent particle, are distinct from functional ultrasound imaging that purely relies on ultrasound to directly image a part of the subject.

For example, the photoexcited mechanoluminescent particle can be contacted with ultrasound in an internal region of the subject, such as the intestines of the patient. In turn, the emission of light from the particles, i.e. the fluorescent light, can be measured in order to image the region. In some cases, the device measuring the fluorescent light is outside the patient, and in other cases it is inside the patient. For example, a fluorescent light measuring device can be inserted into the gastrointestinal tract, e.g. the intestines, of the patient, and can thereby measure the objects or features between the ultrasound stimulated mechanoluminescent particles and the detection device. In such cases, the mechanoluminescent particles can be administered to the subject in any suitable area. For example, if the intestines are to be imaged, the mechanoluminescent particles can be administered in the leg, and optionally photoexcited in the leg, where after they travel through veins to an intestine region, at which they are contacted with ultrasound signal.

### Gene Editing

As the methods allow for light emission deep within the body, the methods can be described as deep-tissue gene editing mediated by ultrasound. These methods, which employ mechanoluminescent particles, are distinct from nonspecific gene editing that purely relies on systemically delivered CRISPR-Cas9 for editing the genome without spatiotemporal precision in the body.

As such, in some cases the tissue comprises a group of compounds that causes genetic modification to the tissue after absorbing the emitted light. In such cases, the method can be referred to as a method of selectively genetically modifying tissue by selectively applying an ultrasound signal to the tissue, i.e. surrounding tissues that do not receive the ultrasound signal or that do not comprise photoexcited mechanoluminescent particles are not genetically modified because they do not receive the light emitted by the particles. The method can also be referred to as light-inducible gene editing. The gene editing can be referred to as spatially-selective gene editing since the gene editing will only occur in locations where the emitted light can penetrate.

For example, the photoexcited mechanoluminescent particle can be contacted with ultrasound in an internal region of the subject, such as the liver or the brain of the patient. In turn, the emission of light from the particles can be used to control the function of photoswitchable Cas9 in order to activate the CRISPR-Cas9 system for localized gene editing. In this case, the mechanoluminescent particles can be administered to the subject via intravenous injection. For example, if the genome in the liver is to be edited, the mechanoluminescent particles can be administered in the leg, and optionally photoexcited in the leg, where after they travel through veins to the liver, at which they are contacted with ultrasound signal to produce localized light emission and gene editing. Examples of such a photoswitchable genome editing methods are described by Moroz-Omori et al (ACS Central Science, 2020, 6, 5, 695, doi:10.1021/acscentrasci.9b01093) and Zhou et al (ACS Chemical Biology, 2018, 13, 2, 443, doi:10.1021/acschembio.7b00603), which are incorporated herein by reference.

### Composition of Mechanoluminescent Particle

Any suitable mechanoluminescent particle can be employed in the methods described herein. In some cases, the mechanoluminescent particle is nanoparticle. In some cases, the mechanoluminescent particle has a biocompatible coating, e.g. a biocompatible coating comprising poly(ethylene glycol). The method typically involves many mechanoluminescent particles, such as 100 or more, 1,000 or more, 10,000 or more, or 100,000 or more. In some cases, the mechanoluminescent particle is a sphere, i.e. it has a spherical shape or it is approximately spherical in shape. In other cases, the mechanoluminescent particle is rod-like or oval-like. In some cases, the mechanoluminescent particle has a diameter ranging from 10 to 500 nm, such as from 30 nm to 250 nm.

In some cases, the mechanoluminescent particle comprises an inorganic material, i.e. it is an inorganic particle, e.g. an inorganic nanoparticle. In some cases, the inorganic nanoparticle comprises zinc sulfide (ZnS); tridymite (X¹Al₂O₄ wherein X¹ is Sr, Ca, Ba, or a combination thereof; melilite (X²₂ESi₂O₇ wherein X² is Ca, Sr, Ba, or a combination thereof, and E is Mg); SrMg₂(PO₄)₂; perovskite (Ba₁₋ₓCaₓTiO₃ wherein 0.25<x<0.8); BaSi₂O₂N₂; SrSi₂O₂N₂; or CaZr(PO₄)₂. Exemplary inorganic mechanoluminescent materials can be found, for example, in Feng et al (Materials, 2018, 11, 484, doi:10.3390/ma11040484, which is incorporated herein by reference.

In some cases, the inorganic nanoparticle comprises zinc sulfide (ZnS), e.g. wurtzite zinc sulfide. In some cases, the zinc sulfide nanoparticle comprises a core-shell structure, e.g. wherein the shell is an un-doped shell and the core is doped with a first and second dopant. In some cases, the core allows for mechanoluminescent properties while the shell, which is of a different material, inhibits quenching before stimulation by ultrasound. In some cases, one dopant provides for absorption and storage of the photoexcitation, while the other dopant provides for photoemission upon mechanical stimulation. In some cases, the first dopant is Co²⁺ and the second dopant is Ag⁺, e.g. in amounts of 0.001% to 1% for each dopant.

In some embodiments, the particle comprises melilite (X²₂ESi₂O₇ wherein X² is Ca, Sr, Ba, or a combination thereof, and E is Mg). For instance, the melilite can have the formula Sr₂MgSi₂O₇. In some cases, the melilite is doped with Eu²⁺, Dy³⁺, or a combination thereof, e.g. wherein each dopant is present at 0.001 mol % to 5 mol %. In some cases, the average diameter of the particles ranges from 5 nm to 100 nm, such as from 15 nm to 50 nm.

Additional examples of mechanoluminescent particles that can be used in the present methods are described in Kersemans et al (Applied Physics Letters, 2015, 107, 234102, doi:10.1063/1.4937354), Feng et al (Materials, 2018, 11(4), 484, doi:10.3390/ma11040484), Terasaki et al (Catalysis Today, 2013, 201, 203, doi:10.1016/j.cattod.2012.04.040), Xu et al (Chem. Sci., 2015, 6, 3236, doi:10.1039/C5SC00466G), and Peng et al (ChemPlusChem, 2015, 80, 1209, doi:10.1002/cplu.201500185), which are incorporated herein by reference.

In some cases, the mechanoluminescent particle comprises an organic material, i.e. it is an organic particle, e.g. an organic particle. Exemplary inorganic materials for mechanoluminescent can be found, for example, in Li et al, Transient and Persistent Room-Temperature Mechanoluminescence from a White-Light-Emitting AIEgen with Tricolor Emission Switching Triggered by Light, Angewandte Chemie International Edition, 2018, 57, 22, 6449-6453, doi: 10.1002/anie.201800762, which is incorporated herein by reference. In some cases, the mechanoluminescent particle comprises N-(4-trifluoromethylphenyl)phthalimide. In some cases, the mechanoluminescent particle comprises 5-fluoro-2-(4-(trifluoromethyl)phenyl)isoindoline-1,3-dione or 5-bromo-2-(4-(trifluoromethyl)phenyl)isoindoline-1,3-dione.

The mechanoluminescent particle are generally down-conversion particles, wherein the photon of light emitted upon mechanical stimulation has a lower energy (i.e. longer wavelength) than the photon of light that photoexcited the mechanoluminescent particle. Stated in another manner, the mechanoluminescent particle is generally not an upconverting particle, i.e. wherein two or more photons are absorbed, and the emitted photon has a higher energy (i.e. shorter wavelength) than either of the two or more absorbed photons. Down-conversion and up-conversion are also referred to as Stokes type emission and anti-Stokes type emission.

The mechanoluminescent particle can absorb and be photoexcited by wavelengths of light that are strongly absorbed by tissue, e.g. wavelengths ranging from 350 nm to 500 nm.

After photoexcitation, the mechanoluminescent particles can be stimulated by ultrasound with frequencies in the range of 100 kHz to 15 MHz, emitting mechanoluminescence with wavelengths ranging from 400 nm to 1700 nm.

### Systems

Provided are systems for contacting a tissue of a subject with light, wherein each system comprises: an ultrasound device configured to apply an ultrasound signal to a mechanoluminescent particle in proximity to the tissue, thereby causing the mechanoluminescent particle to emit light that contacts the tissue. The ultrasound emitting device can have any of the features or properties described above, e.g. it can have a frequency ranging from 150 kHz to 15 MHz and it can be focused ultrasound.

In some cases, the system further includes an apparatus comprising a nucleic acid comprising a nucleotide sequence encoding the photosensitive protein, e.g. the photosensitive protein that can be used for optogenetics, as described above. For instance, the apparatus can be a syringe that contains an aqueous liquid comprising the nucleic acid.

In some cases, the system further includes the mechanoluminescent particle, which can have any of the features described above, e.g. it can comprise zinc sulfide (ZnS) or melilite (X²₂ESi₂O₇ wherein X² is Ca, Sr, Ba, or a combination thereof, and E is Mg).

### Kits

Provided are kits that can be employed to perform the methods described herein. In some cases, the kit includes two or more of: a mechanoluminescent particle; a device for administering the mechanoluminescent particle to a subject; a device for emitting light onto an external surface of a subject such that a mechanoluminescent particle in the subject is photoexcited; and a device for emitting ultrasound. In some cases, the kit includes the mechanoluminescent particle and the device for emitting ultrasound. In some cases, the kit further includes a device for emitting light onto an external surface of a subject such that a mechanoluminescent particle in the subject is photoexcited, a device for administering the mechanoluminescent particle to a subject, or both.

Notwithstanding the appended claims, the disclosure is also defined by the following clauses:
1. A method of contacting a tissue of a subject with light, comprising:
   applying an ultrasound signal to a photoexcited mechanoluminescent particle while the mechanoluminescent particle is in proximity to the tissue of the subject, thereby causing the mechanoluminescent particle to emit light that contacts the tissue.
2. The method of clause 1, wherein the tissue comprises a neuron that comprises a photosensitive protein, and wherein the emitted light modulates the photosensitive protein.
3. The method of clause 2, wherein the modulation results in hyperpolarization of the neuron.
4. The method of clause 2, wherein the modulation results in depolarization of the neuron.
5. The method of any one of clauses 2-4, further comprising genetically modifying the neuron to express the photosensitive protein.
6. The method of any one of clauses 2-5, wherein the photosensitive protein is a channelrhodopsin-2 (ChR2), a Volvox carteri light-activated protein (VChR1), a iC++, a ChRmine, or a halorhodopsin (HPHR).
7. The method of clause 6, wherein the photosensitive protein is a halorhodopsin.
8. The method of clause 7, wherein the halorhodopsin is a halorhodopsin from Natronomonas (NpHR).
9. The method of clause 1, wherein the tissue comprises a group of compounds that causes genetic modification to the tissue after absorbing the emitted light.
10. The method of clause 9, wherein the group of compounds comprises a CRISPR compound and a Cas9 compound.
11. The method of clause 1, further comprising fluorescently imaging the tissue by measuring the light emitted by the mechanoluminescent particle.
12. The method of clause 11, wherein the measuring comprises measuring light with a light measurement device inserted into the gastrointestinal tract of the subject.
13. The method of clause 1, wherein the tissue comprises a photosensitizer that is contacted by the emitted light, thereby generating a reactive oxygen species.
14. The method of clause 13, wherein the subject has a disease and the method is a method of treating the subject for the disease by generating a reactive oxygen species in the tissue.
15. The method of clause 14, wherein the disease is an infection by a microorganism.
16. The method of clause 14, wherein the disease is cancer and the tissue comprises cancer cells.
17. The method of any one of clause 1-16, further comprising administering the mechanoluminescent particle to the subject.
18. The method of clause 17, wherein the administering comprises intravenously injecting a liquid that comprises the mechanoluminescent particle into the subject.
19. The method of any one of clauses 1-18, further comprising photoexciting the mechanoluminescent particle before the application of the ultrasound signal.
20. The method of clause 19, wherein the photoexciting comprises applying excitation light to the mechanoluminescent particle while the particle is outside the subject.
21. The method of clause 19, wherein the photoexciting comprises applying excitation light to an external surface of the subject after administering the mechanoluminescent particle to the subject such that the excitation light photoexcites the mechanoluminescent particle.
22. The method of clause 21, wherein the external surface is skin covering a blood vessel and the mechanoluminescent particle is in the blood vessel during the application of light.
23. The method of clause 22, wherein the blood vessel is 20 mm or less below the skin.
24. The method of any one of clauses 21-23, wherein the external surface of the subject is part of the head or the neck of the subject.
25. The method of any one of clauses 22-24, wherein the blood vessel is a facial artery or a jugular vein.
26. The method of any one of clauses 19-25, further comprising administering the mechanoluminescent particle to the subject, wherein the time between the administering and the photoexciting is 60 minutes or less.
27. The method of any one of clauses 20-26, wherein 80% or more of the photons of the excitation light have a wavelength ranging from 350 nm to 450 nm.
28. The method of any one of clauses 1-27, wherein 80% or more of the photons of the light emitted by the mechanoluminescent particle have a wavelength ranging from 400 nm to 1700 nm.
29. The method of any one of clauses 1-28, wherein the mechanoluminescent particle is a nanoparticle having a dimension ranging from 1 nm to 1 μm.
30. The method of clause 29, wherein the dimension ranges from 30 nm to 250 nm.
31. The method of any one of clauses 1-30, wherein the mechanoluminescent particle is a sphere.
32. The method of any one of clauses 1-31, wherein the mechanoluminescent particle comprises an inorganic material.
33. The method of clause 32, wherein the mechanoluminescent particle comprises a semiconductor material, an insulator material, or both, wherein at least one of the semiconductor material and the insulator material has a bandgap energy ranging from 2 eV to 5 eV.
34. The method of any one of clauses 1-33, wherein the mechanoluminescent particle comprises zinc sulfide (ZnS); tridymite (X¹Al₂O₄ wherein X¹ is Sr, Ca, Ba, or a combination thereof); melilite (X²₂ESi₂O₇ wherein X² is Ca, Sr, Ba, or a combination thereof, E is Mg); SrMg₂(PO₄)₂; perovskite (Ba₁₋ₓCaₓTiO₃ wherein 0.25<x<0.8); BaSi₂O₂N₂; SrSi₂O₂N₂; CaZr(PO₄)₂; or a combination thereof.
35. The method of clause 34, wherein the mechanoluminescent particle comprises zinc sulfide (ZnS).
36. The method of clause 35, the zinc sulfide is wurtzite zinc sulfide.
37. The method of any one of clauses 1-36, wherein the mechanoluminescent particle is a core-shell particle comprising an un-doped shell and a core doped with a first and second dopants.
38. The method of clause 37, wherein the first dopant is Co²⁺ and the second dopant is Ag⁺.
39. The method of clause 38, wherein the amount of Co²⁺ is 0.001 mol % to 1 mol %.
40. The method of clause 38 or 39, wherein the amount of Ag⁺ is 0.001 mol % to 1 mol %.
41. The method of clause 34, wherein the mechanoluminescent particle comprises melilite (X²₂ESi₂O₇ wherein X² is Ca, Sr, Ba, or a combination thereof, E is Mg).
42. The method of clause 41, wherein the melilite is doped with Eu²⁺, Dy³⁺, or a combination thereof.
43. The method of any one of clauses 1-42, wherein the mechanoluminescent particle comprises an organic material.
44. The method of clause 43, wherein the organic material comprises a N-(4-trifluoromethylphenyl)phthalimide group.
45. The method of any one of clauses 1-44, wherein the mechanoluminescent particle comprises a biocompatible coating.
46. The method of clause 45, wherein the biocompatible coating comprises polyethylene glycol or a derivative thereof.
47. The method of any one of clauses 1-46, wherein the ultrasound signal is a focused ultrasound signal (FUS).
48. The method of any one of clauses 1-47, wherein the ultrasound signal has a frequency ranging from 150 kHz to 15 MHz.
49. The method of any one of clauses 1-48, wherein the ultrasound signal is repeated at a rate ranging from 0.2 repetitions per second to 5 repetitions per second.
50. The method of any one of clauses 1-49, wherein the ultrasound signal has a spatial peak pulsed average intensity (I_{SPPA}) at a target neuron ranging from 1 W/cm² to 100 W/cm².
51. The method of clause 50, wherein the spatial peak pulsed average intensity at a target neuron ranges from 5 W/cm² to 15 W/cm².
52. The method of any one of clauses 1-51, wherein the time interval between the application of the ultrasound signal and the emission of light from the photoexcited mechanoluminescent particle is 9 ms or less.
53. The method of any one of clauses 1-52, wherein the time interval between the photoexcitation of the mechanoluminescent particles and the application of ultrasound ranges from 1 second to 60 minutes.
54. A system for contacting a tissue of a subject with light, the system comprising:
   an ultrasound device configured to apply an ultrasound signal to a mechanoluminescent particle in proximity to the tissue, thereby causing the mechanoluminescent particle to emit light that contacts the tissue.
55. The system of clause 54, wherein the ultrasound signal is a focused ultrasound signal.
56. The system of any one of clauses 54-55, further comprising the mechanoluminescent particle.
57. The system of any one of clauses 54-56, further comprising an excitation light source that emits excitation light that can photoexcite the mechanoluminescent particle.
58. The system of any one of clauses 54-57, further comprising an apparatus comprising a liquid a nucleic acid comprising a nucleotide sequence encoding for a photosensitive protein.
59. The system of any one of clauses 54-57, further comprising a light measurement device configured to measure the light emitted from the mechanoluminescent particle.
60. The system of clause 59, wherein the light measurement device is configured to be inserted into the gastrointestinal tract of the subject.
61. The system of any one of clauses 54-57, further comprising an apparatus comprising a liquid comprising a photosensitizer that can generate a reactive oxygen species by being contacted with the light emitted by the mechanoluminescent particle.
62. A kit comprising two or more of:
   a mechanoluminescent particle;
   a device for administering the mechanoluminescent particle to a subject;
   a device for emitting light onto an external surface of a subject such that a mechanoluminescent particle in the subject is photoexcited; and
   a device for emitting ultrasound.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); and the like.

### INTRODUCTION

Optogenetics, which uses visible light to control the cells genetically modified with light-gated proteins, is a powerful tool for precise deconstruction of neural circuitry with neuron-subtype specificity. However, due to limited tissue penetration of visible light, invasive craniotomy and intracranial implantation of tethered optical fibers are usually involved for in vivo optogenetic modulation. Here is reported mechanoluminescent nanoparticles that can act as local light sources in the brain when triggered by brain-penetrant focused ultrasound (FUS) through intact scalp and skull. Mechanoluminescent nanoparticles can be delivered into the blood circulation via intravenous injection, recharged by 400-nm photoexcitation light in superficial blood vessels during circulation, and turned on by FUS to emit 470-nm light repetitively in the intact brain for optogenetic stimulation. Unlike the conventional ‘outside-in’ approaches of optogenetics with fiber implantation, this method provides an ‘inside-out’ approach to deliver nanoscopic light emitters via the intrinsic circulatory system and switch them on and off at any time and location of interest in the brain through a minimally invasive ultrasound interface.

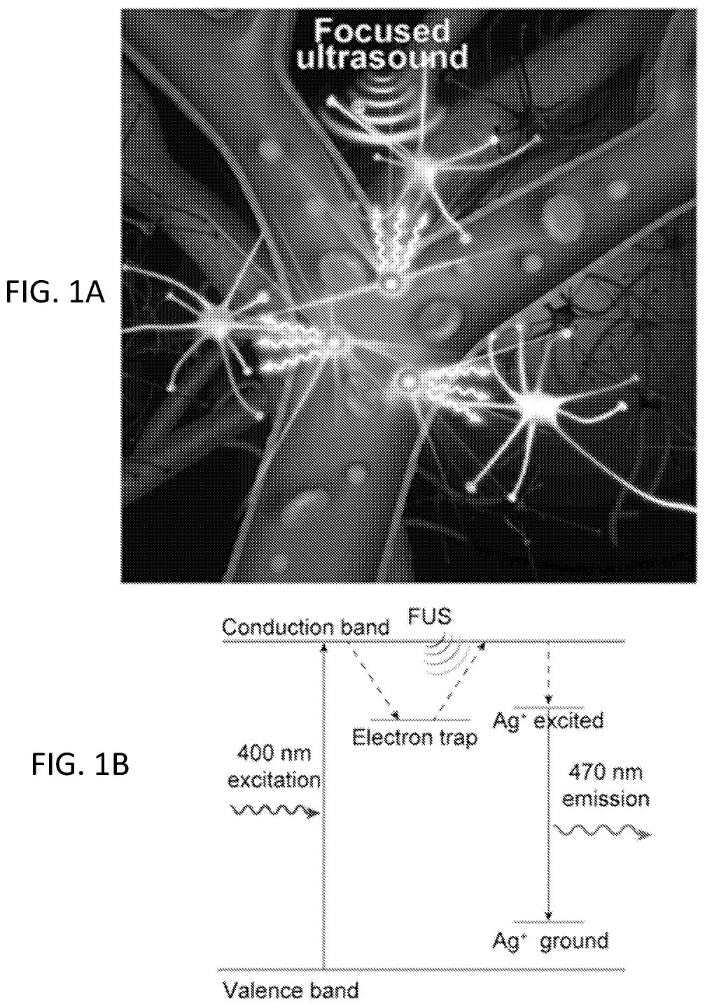

The combination of FUS excitation and intravenous delivery of mechanoluminescent nanoparticles offers a new approach of ‘sono-optogenetics’ (FIG. 1A), affording fiber-free optogenetics through intact scalp and skull in live animals.

### Example 1: Synthesis of ZnS:Ag/Co Nanoparticles

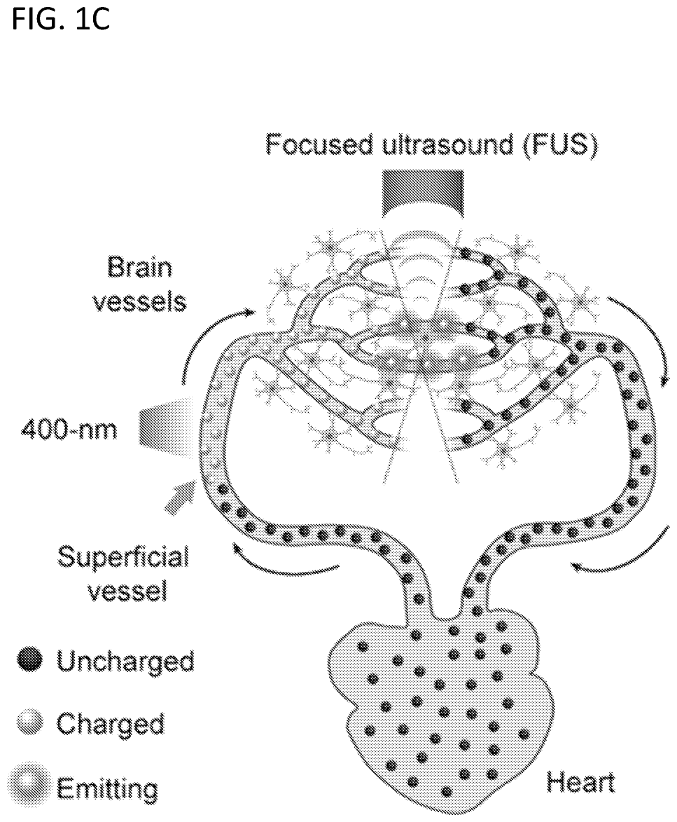

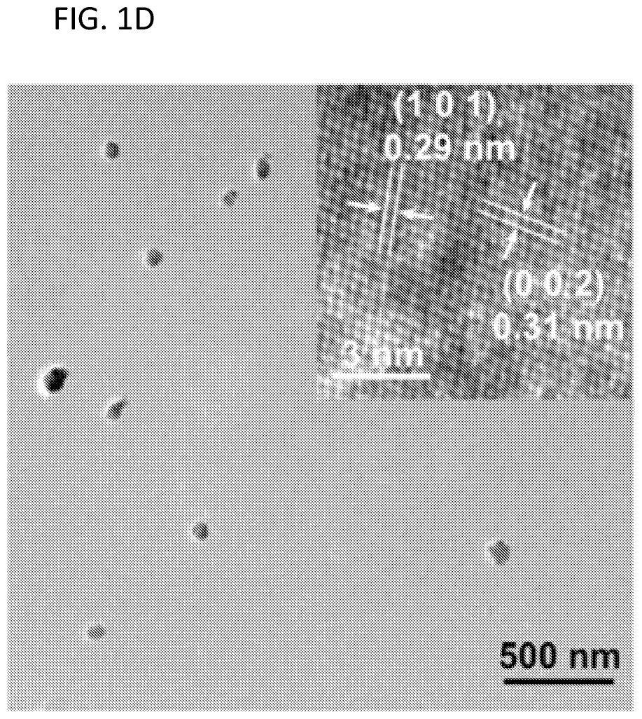

Mechanoluminescent nanoparticles (ZnS:Ag, Co/ZnS) were synthesized via a two-step hydrothermal process (see Methods and Materials below). ZnS is a type II-VI semiconductor with a bandgap energy of 3.7 eV, leading to efficient absorption of 400-nm light and excitation of electrons to the conduction band (FIG. 1B, left; FIG. 2A & FIG. 3). Co²⁺ dopant ions in the ZnS matrix create defect states with a trap depth of 0.5 eV below the conduction band, which trap the excited electrons and act as ‘energy relays’ that store the photoexcitation energy without emission (FIG. 2B). When FUS is applied, the mechanical stress leads to charge separation in the piezoelectric ZnS matrix, effectively tilting the conduction band and making it easier for the trapped electrons to get ‘detrapped’ and return to the conduction band (FIG. 1B, middle & FIG. 2C). After the ‘detrapping’ process, Ag⁺ dopant ions receive the energy transferred from the ‘detrapped’ electrons (FIG. 2D) and produce 470-nm emission characteristic of Ag luminescent centers as previously reported (FIG. 1B, right & FIG. 2E) (33-35). The entire process of photoexcitation, defect-induced trapping, FUS-triggered detrapping, energy transfer and photoemission can be repeated indefinitely in the mechanoluminescent nanoparticles for repetitive optogenetic stimulation.

### Example 2: Testing Properties of ZnS:Ag/Co Nanoparticles

The mechanoluminescent materials described above provide an ‘energy relay’ between the 400-nm photoexcitation and FUS-triggered 470-nm emission, which can be exploited for minimally invasive deep-brain optogenetic neural stimulation via the endogenous blood circulatory system in a living subject (FIG. 1C). Specifically, sono-optogenetics is realized with several key design features on the systemic level of the entire organism. First, due to the poor tissue penetration of 400-nm photoexcitation, uncharged mechanoluminescent nanoparticles need to be close enough to the surface of the skin to receive photoexcitation and become charged with energy. Taking advantage of the circulatory system that has blood vessels distributed across various depths in the body, a 400-nm excitation light source was placed near the facial artery and jugular vein, which are within a depth of ca. 0.8 mm from the surface of skin in mice (36), for charging the circulating mechanoluminescent nanoparticles when they pass through these illuminated blood vessels (FIG. 1C, left). Second, once charged, these nanoparticles store the energy of photoexcitation and can circulate at any depth inside the body, before releasing the stored energy only when triggered by the FUS. Taking advantage of the deep-tissue penetration of FUS, a FUS transducer was used with a center frequency of 1.5 MHz to provide minimally invasive, localized ultrasound stimulation of circulating nanoparticles as they flow past the focus of the applied ultrasound and emit 470-nm light for optogenetic neural stimulation as a result of the mechanoluminescence process (FIG. 1C, top). Third, the constant blood circulation of the body provides continuous supply of charged nanoparticles to the ultrasound focus in the brain for repetitive optogenetic stimulation within the circulation lifetime of the nanoparticles.

Despite the promise of circulation delivered rechargeable nanoparticles for minimally invasive optogenetic neuromodulation, one of the challenges faced was the relatively low intensity of mechanoluminescence from the ZnS:Ag, Co nanoparticles (37), which was ca. one order of magnitude below the threshold of efficient optogenetic stimulation of ChR2 (38). To enhance the emission of ZnS:Ag, Co nanoparticles, the ZnS:Ag, Co nanoparticles were coated with an additional ZnS layer to prevent the luminescence quenching effect caused by the solvent or ligand molecules (39, 40). Comprehensive studies were carried out of morphological, size, structural and spectral characterizations of ZnS:Ag, Co/ZnS nanoparticles, in comparison with the uncoated ZnS:Ag, Co nanoparticles and undoped ZnS nanoparticles for the purpose of luminescence enhancement, with several key findings.

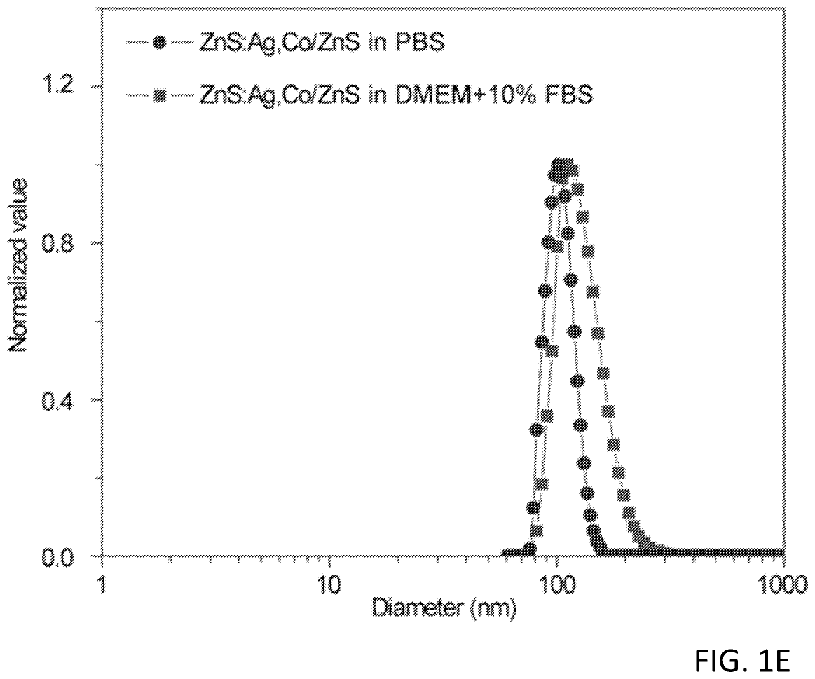

First, ZnS:Ag, Co/ZnS nanoparticles have a spherical-like morphology with an average diameter of 86.6±13.0 nm (mean±standard deviation; FIG. 1D). High resolution TEM imaging (FIG. 1D, inset) and XRD spectroscopy (FIG. 4) revealed the nanoparticles as wurtzite ZnS. It is noteworthy that wurtzite has a high piezoelectric coefficient due to its non-centrosymmetric structure, leading to stronger mechanoluminescence than zinc-blende with a low piezoelectric coefficient (35). To render these nanoparticles biocompatible, surface modification was used with an amphiphilic coating of 1,2-distearoyl-sn-glycero-3-phosphoethanolamine-N-[methoxy(polyethylene glycol)-2000] (DSPE-mPEG, see Materials and Methods). As indicated by the dynamic light scattering (DLS) measurements (FIG. 1E), the surface-modified ZnS:Ag, Co/ZnS nanoparticles showed good monodispersity with an average hydrodynamic diameter of 101.4 nm in phosphate buffered saline (PBS) and 111.5 nm in cell medium, in good agreement with the TEM imaging result and the radius of gyration of PEG chains (41).

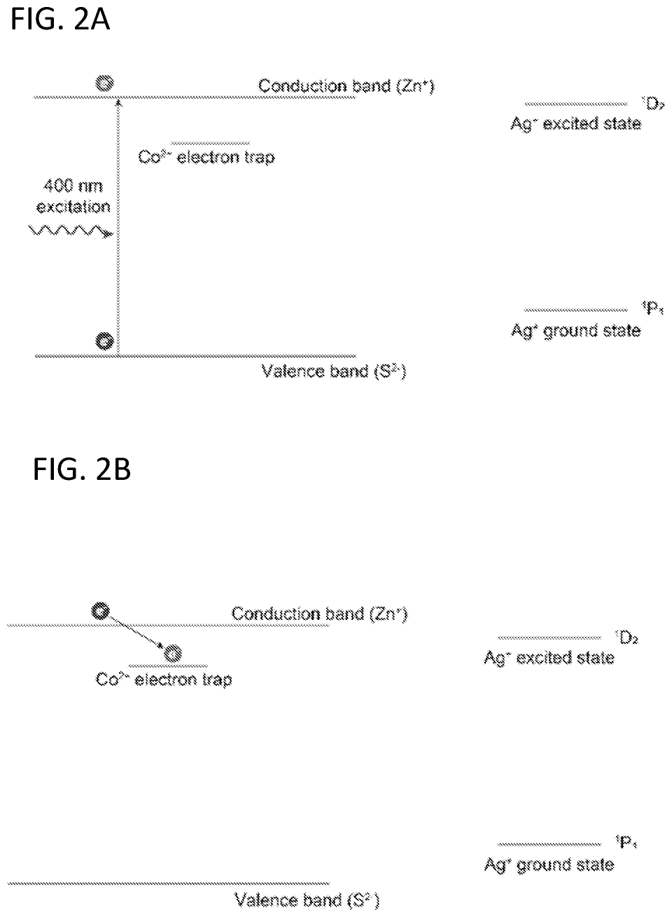

Second, it was found a 8.6-fold increase of photoluminescence (FIG. 5, panel A) and a 9.2-fold increase of mechanoluminescence (FIG. 5, panel B) for ZnS:Ag, Co/ZnS nanoparticles compared to the uncoated ZnS:Ag, Co nanoparticles, suggesting effective enhancement of the mechanoluminescence of ZnS:Ag, Co/ZnS nanoparticles that makes them suitable for optogenetic activation of ChR2. The spectra of photoluminescence and mechanoluminescence are found to perfectly overlap with each other after normalization, with the main emission peak located at 470 nm for optimal photoactivation of ChR2, which has a highly overlapping spectral profile of absorbance (FIG. 5, panels C and D). In contrast, the undoped ZnS nanoparticles exhibited blue-shifted photoluminescence with a center wavelength of 430 nm and no detectable mechanoluminescence, suggesting the role of Ag⁺ dopant ions in tuning the emission wavelength to match the absorption profile of ChR2 and the role of Co²⁺ dopant ions to create defect energy states for storing absorbed photoexcitation energy.

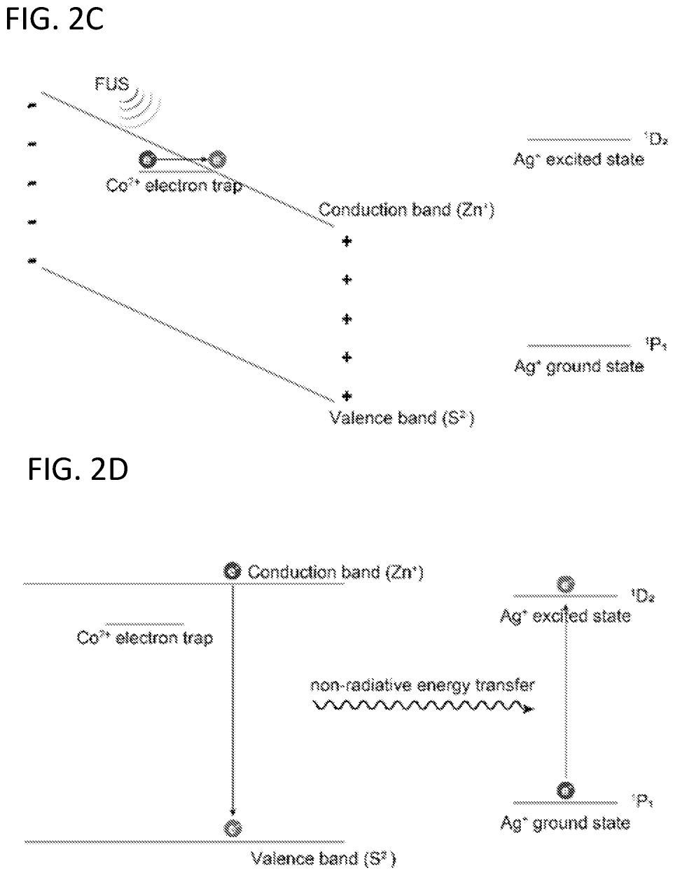

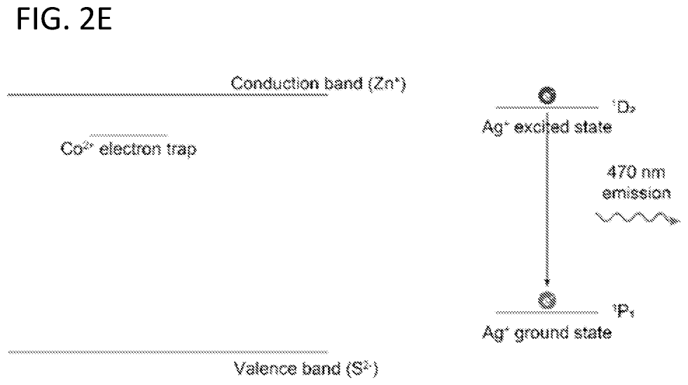

The enhanced light emission at 470 nm from ZnS:Ag, Co/ZnS nanoparticles was bright enough to be visualized under ambient lighting conditions under FUS excitation (FIG. 6). Time-resolved luminescence measurement revealed an 18-fold increase of luminescence intensity upon FUS excitation from the afterglow decay baseline of ZnS:Ag, Co/ZnS nanoparticles (FIG. 7).

### Example 3: Testing Nanoparticle Properties in an Artificial Body

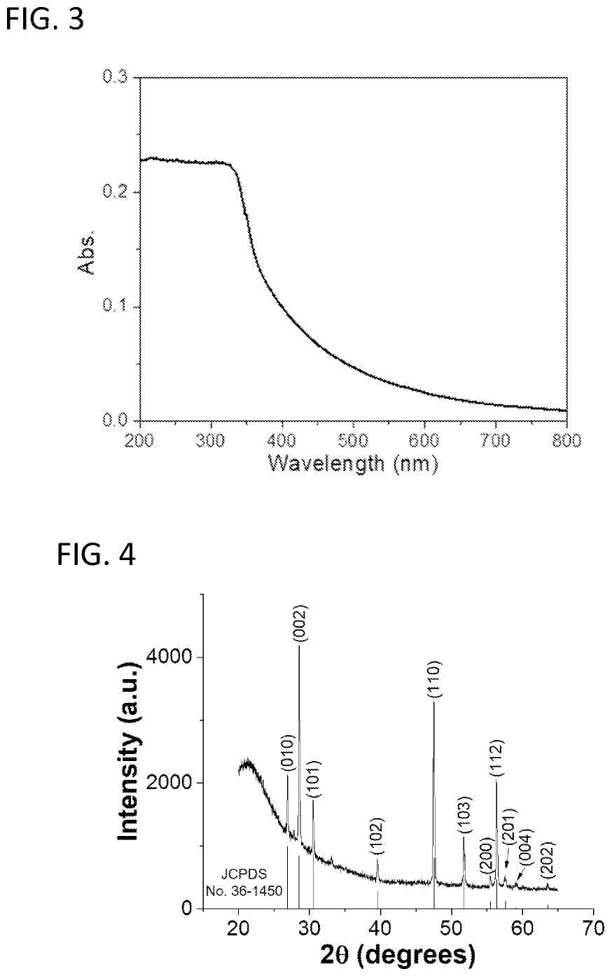

To test the feasibility of using them as rechargeable light sources in vivo, an artificial circulatory system was made to produce localized photoirradiation in a repeatable and controllable manner (FIG. 5, panel D and FIG. 8). The artificial circulatory system comprises several key components to mimic the systemic circulation in a live animal. First, a cylindrical piece of polydimethylsiloxane (PDMS) was used to mimic the biological tissue, with a tunnel inside the PDMS phantom to mimic a deep blood vessel in the brain. Second, both ends of the PDMS-embedded tunnel were connected by a closed loop of Tygon tubing, which was filled with ZnS:Ag, Co/ZnS nanoparticle suspension to mimic the blood stream that carries circulation-delivered light source. Third, a 400-nm light source was placed next to the Tygon tubing without illuminating any part of the PDMS phantom (see Materials and Methods) to recharge the circulating nanoparticles before they enter the PDMS-embedded tunnel and release the stored energy. Fourth, a FUS transducer was placed over the PDMS phantom to excite the circulating nanoparticles inside the tunnel, with a fiber optic cannula inserted to the opposite end of the PDMS phantom for localized light intensity measurement. Finally, a peristaltic pump was used to mimic the heart that drives the circulation at a constant speed, pumping the charged nanoparticles into the ‘brain vessel’ model for FUS-triggered light emission and the discharged ones back into the circulation for recharging.

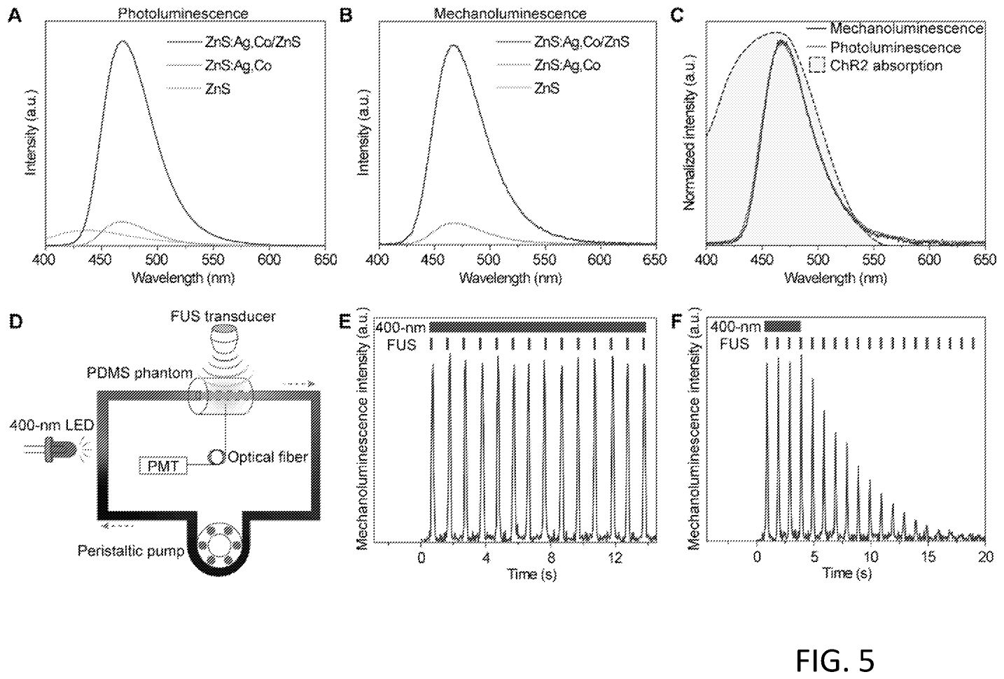

The local light emission was measured from circulating nanoparticles in real time when FUS was applied with a repetition rate of 1 Hz with the following findings. First, FUS pulses triggered immediate light emission with a short delay of ˜4 ms (FIG. 9), while the baseline intensity in the absence of FUS was below the detection limit of the spectrophotometer with only noise fluctuations. This delay time is shorter than the reported time-to-spike latency time of ChR2 for neural stimulation, the latter of which is above 10 ms (5), and thus does not impose significant delay to the millisecond temporal precision of optogenetic stimulation. Second, repeated FUS excitation demonstrated stable peak intensity of emitted light, in which the variation was found to be within 5% of the peak intensity (FIG. 5, panel E), when the 400-nm photoexcitation light source was kept on to continuously charge the nanoparticles in the circulation. This suggests that a steady state of charging by photoexcitation and discharging by FUS was reached for the light source in the circulatory system. Third, when the 400-nm photoexcitation light source was turned off while keeping all other components in the circulatory system unchanged, it was found a rapid decrease of the peak intensity of the FUS-triggered mechanoluminescence (FIG. 5, panel F), suggesting depletion of the stored energy in the circulating nanoparticles over time. These results suggest that the circulatory system was sufficient to deliver charged nanoparticles to the ultrasound focus for repeatable light emission.

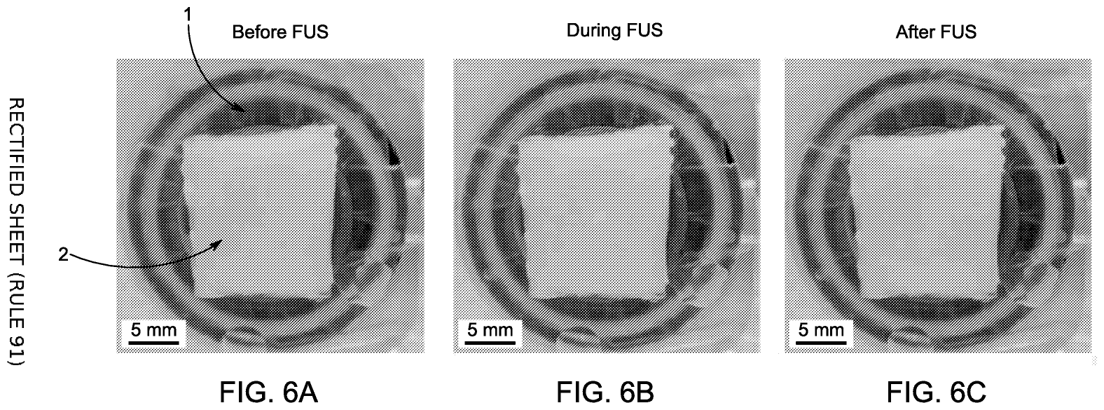

It was then considered if the artificial circulatory system could be used to evoke action potentials from spiking cells expressing ChR2 under repetitive FUS stimuli. NaV 1.3 KIR 2.1 human embryonic kidney (HEK) cells (42) transfected with ChR2 and cultured in a petri dish were used for this study. Since the cell medium was stationary and not circulating for replenishment of charged nanoparticles, it was assembled an artificial circulatory system to provide continuous 470-nm emission by placing the tubing of the circulatory system between the cell culture and the FUS transducer (see Materials and Methods). The tubing, which was placed next to the cell culture in this system, acted as the light source for optogenetic stimulation of ChR2 in spiking HEK cells (FIG. 10A). It was hypothesized that the spiking HEK cells would be triggered by FUS to fire action potentials in synchrony with timed FUS pulses only when the artificial circulatory system was filled with mechanoluminescent nanoparticles and was constantly charged with the 400-nm photoexcitation. The results of the experiments confirmed the hypothesis with the following key findings. First, extracellular recordings with a microelectrode array (MEA) revealed periodic single-unit action potentials only in the ChR2(+) and nanoparticle (+) group with FUS excitation, in which the spiking HEK cells expressed ChR2 and the artificial circulatory system had circulating mechanoluminescent nanoparticles providing constant 470-nm light emission under FUS (FIG. 10B). In contrast, all other groups demonstrated minimal firing activity with FUS excitation, suggesting the lack of direct FUS activation of ChR2. Second, the overlay of triggered action potentials under >80 consecutive FUS stimuli revealed typical waveforms of extracellular single-unit spikes as negative peaks (FIG. 10C). The spike amplitude was stably measured under repetitive FUS stimulation, showing a statistically significant difference between the ChR2(+) and nanoparticle (+) group and all other control groups (FIG. 10D).

### Example 4: Testing Nanoparticle Properties In Vivo

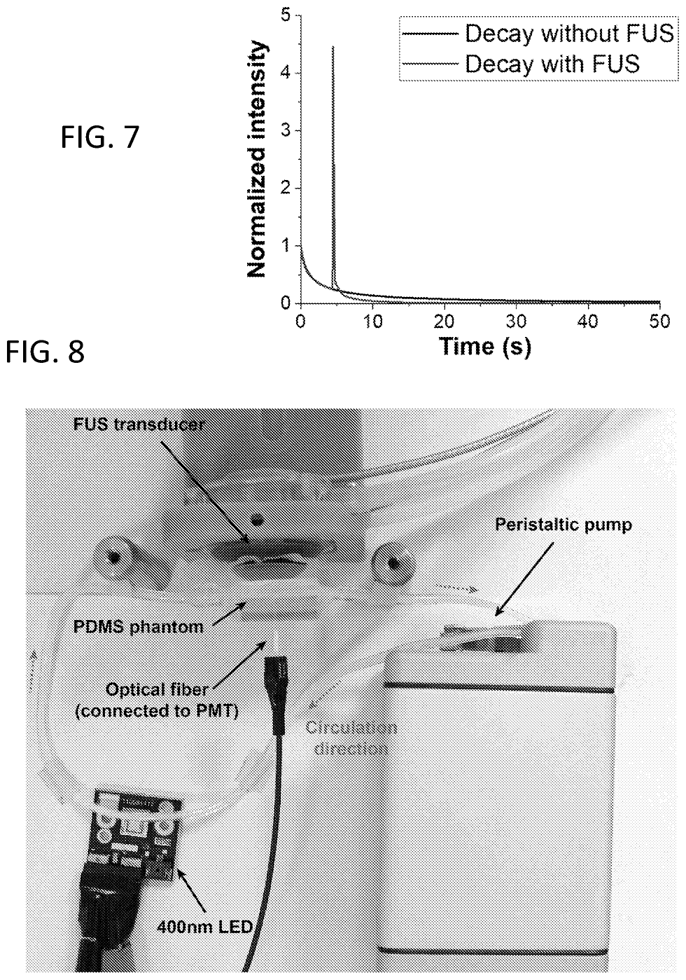

Having demonstrated rapid, reproducible optogenetic activation of ChR2 in vitro, it was hypothesized if ZnS:Ag, Co/ZnS nanoparticles in the intrinsic circulatory system in live animals allowed for optogenetic stimulation of ChR2-expressing neurons in the brain without craniotomy or any brain implant. It was hypothesized that owing to the deep tissue penetration of FUS and the densely distributed cerebral vasculature in the mouse brain, circulation-delivered ZnS:Ag, Co/ZnS nanoparticles could act as localized light sources by providing sufficient 470-nm emission to stimulate ChR2-expressing neurons located in the vicinity of blood vessels, thus allowing for optogenetic stimulation of the brain through intact scalp and skull (FIG. 11A). To demonstrate the proof of concept of in vivo sono-optogenetic stimulation, a Thy1-ChR2-YFP mouse was positioned under anesthesia in a stereotaxic frame, exposing the intact scalp in direct contact to an FUS transducer (FIG. 11B). The intensity of 470-nm luminescence emitted was first measured from circulating nanoparticles under repetitive FUS excitations and continuous 400-nm recharging light (see Materials and Methods) to ensure the intensity was sufficient to activate ChR2 in the brain.

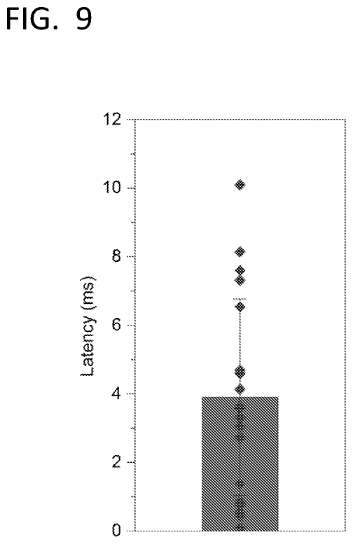

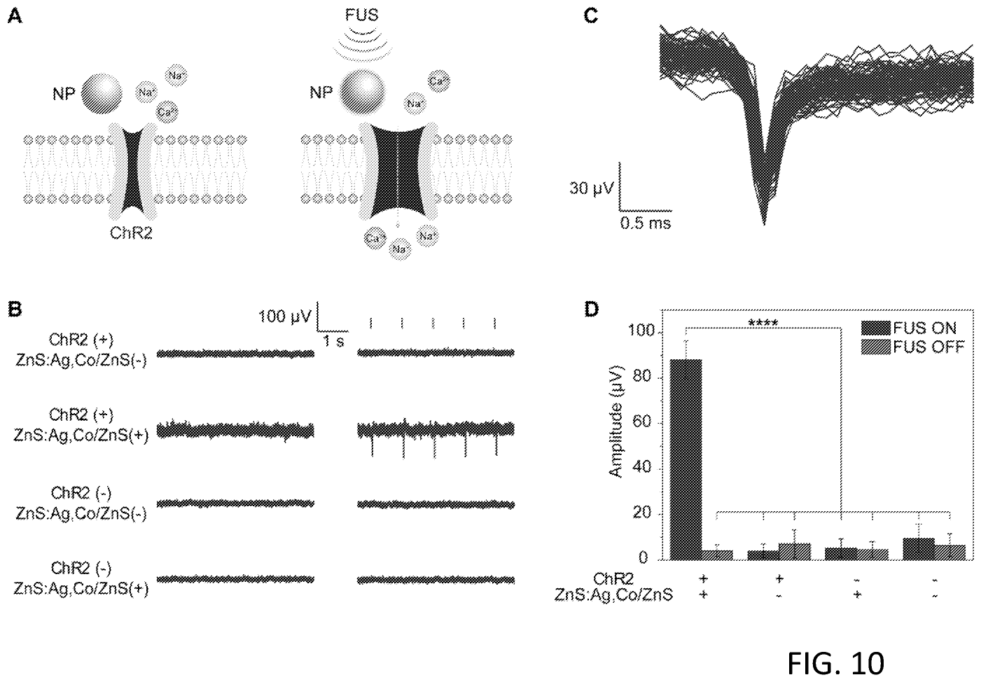

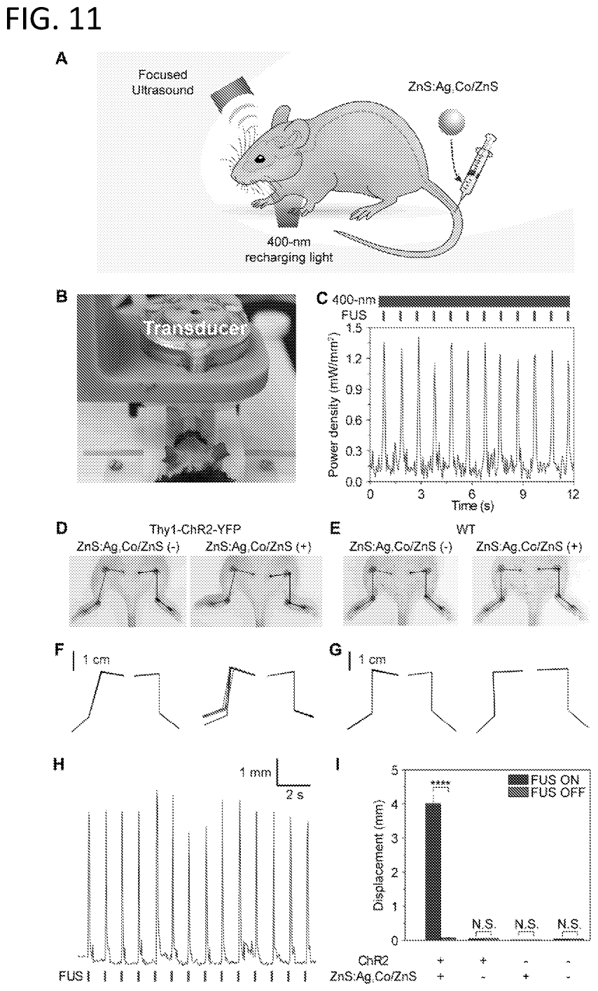

The in vivo luminescence measurements revealed several key findings. First, intravenously injected ZnS:Ag, Co/ZnS nanoparticles at a concentration of 5 mg/mL in the blood circulation produced 470-nm light emission under FUS excitation, with an equivalent power density of ca. 1.2 mW/mm² (FIG. 11C) compared to an intracranially implant fiber cannula. It has been reported that wild-type ChR2 can be activated with >50% spiking probability under this power density with direct fiber illumination (38). A measured circulation half-life of 127.8±45.3 min suggested that the concentration of circulating ZnS:Ag, Co/ZnS nanoparticles in blood stream stayed above 80% of the initial concentration for the first half hour after intravenous injection (FIG. 12). Second, the peak power density of 470-nm mechanoluminescence measured in the brain remained stable over repetitive FUS stimuli, owing to the 400-nm LED positioned near the neck to recharge the circulating nanoparticles in the superficial vessels. Third, the FUS stimuli in the in vivo experiments were measured to have a spatial peak pulsed average intensity (I_{SPPA}) of 10 W/cm² at the ultrasound focus in the brain tissue (FIG. 13), significantly lower than the safety limit of FUS in mice (43) and the threshold involved to provide non-specific neural stimulation at 1.5 MHz (44). It was estimated that with the protocol, the pressure produced by FUS in the brain tissue was able to produce ‘band tilting’ of 0.96 V, sufficient to release the trapped electrons at a depth of 0.5 V to the conduction band for mechanoluminescence emission (35). Fourth, the temperature increase in the local brain tissue with continuous sono-optogenetic stimulation was found to be <0.2° C. over 10 s (FIG. 14), suggesting negligible intracranial heating that would otherwise alter neuronal physiology due to temperature changes (45). These findings suggested the feasibility of using relatively low-power, through-scalp FUS to activate ChR2-expressing neurons and evoke behavioral responses in live animals via circulation-delivered nanoparticles.

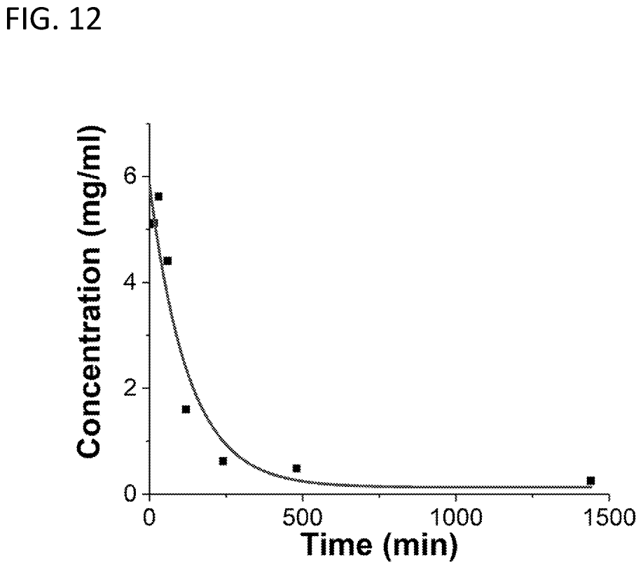

Using the parameters determined by the light intensity measurement above, it was demonstrated activation of unilateral limb movement by focusing ultrasound to the secondary motor cortex (M2) subserved by blood circulation carrying charged ZnS:Ag, Co/ZnS nanoparticles in the right hemisphere through intact scalp and skull. A video camera was used to track the kinematics of the contralateral and ipsilateral limbs, both of which were marked with dots of different colors at the joints. The experiments have revealed key results that demonstrated unilateral limb activation via sono-optogenetic stimulation. First, the Thy1-ChR2-YFP mouse with ChR2 expressed in the M2 demonstrated obvious hindlimb motion, which was synchronized with the FUS excitation, after injection of ZnS:Ag, Co/ZnS nanoparticles in the blood circulation. The same animal did not exhibit any hindlimb motion before nanoparticle injection under the same stimulation protocol (FIG. 11D). Second, the wild-type mouse without ChR2 expression in the brain demonstrated no hindlimb motion, regardless of the presence of mechanoluminescent nanoparticles in the blood circulation (FIG. 11E). These results suggested that FUS stimuli alone were unable to evoke limb movement at the specific frequency and power density used in the experiments. It has been reported that ultrasound can stimulate neurons in the brain by modulation of endogenous mechanosensitive proteins (24, 46); the finding of minimal limb motion with ultrasound alone was likely due to the relatively low power density of FUS at the frequency of 1.5 MHz (44). Third, hindlimb kinematics analysis revealed unilateral motion only in the left hindlimb of ChR2 mouse in the presence of mechanoluminescent nanoparticles, which was contralateral to the focus of ultrasound in the right hemisphere (FIG. 11F-G). The ipsilateral hindlimb showed minimal motion under FUS excitation (FIG. 15). Fourth, quantitative analysis of the contralateral hindlimb kinematics revealed reproducible range of motion over repetitive FUS stimuli (FIG. 11H), which is consistent with the stable peak intensity of light emission discussed above (FIG. 11C). Finally, the results were successfully reproduced in a group of three Thy1-ChR2-YFP mice, with statistically significant difference in comparison with any of the control groups that did not receive injection of ZnS:Ag, Co/ZnS nanoparticles, did not have ChR2 in the brain, or did not have either (FIG. 11I).

### Example 5: Synthesis and Testing of Sr₂MgSi₂O₇:Eu²⁺, Dy³⁺ Nanoparticles

Sr₂MgSi₂O₇:Eu²⁺, Dy³⁺ nanoparticles were prepared by a two-step synthesis. In the first step, Sr₂MgSi₂O₇:Eu²⁺, Dy³⁺ bulk materials were synthesized by a high-temperature solid-state reaction. Briefly, the stoichiometric ratio of SrCO₃, Eu₂O₃, Dy₂O₃, (MgCO₃)₄Mg(OH)₂.5H₂O and SiO₂ were weighed as the raw materials and then ground by mortar for 1 h, followed by annealing at 1050° C. under a reducing gas flow of N₂ (95 v %) and H₂ (5 v %) for 2 h. The doping ratios of Eu³⁺ and Dy³⁺ were tuned from 0.001 mol % to 5 mol %. Secondly, the as-prepared Sr₂MgSi₂O₇:Eu²⁺, Dy³⁺ bulk materials were added into a mixed solution of sodium citrate and citric acid and stirred for 24-96 h. The Sr₂MgSi₂O₇:Eu²⁺, Dy³⁺ nanoparticles can be precipitated by centrifugation.

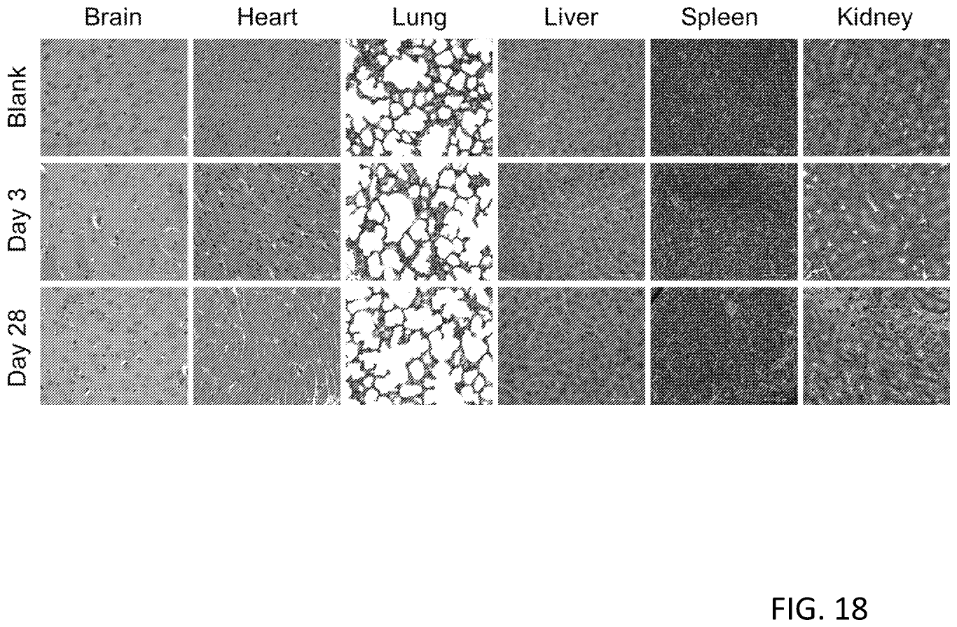

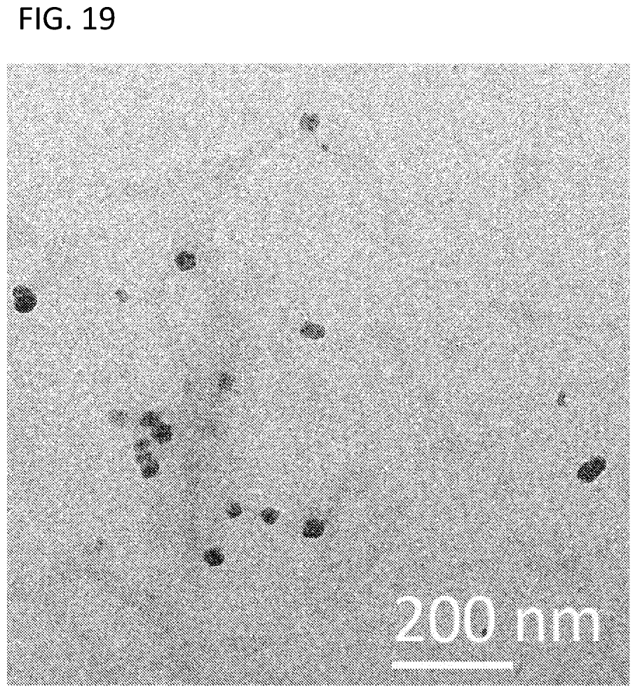

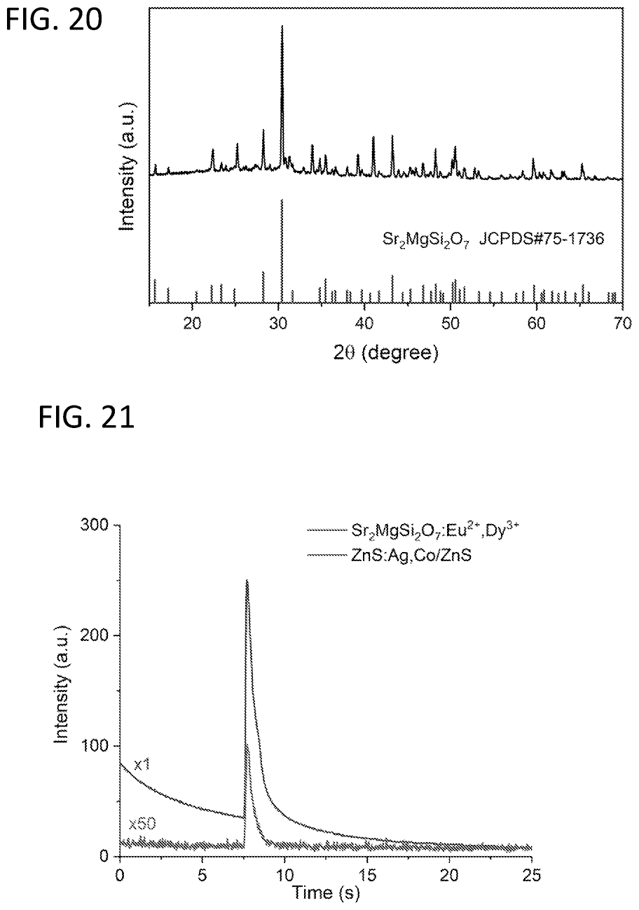

Sr₂MgSi₂O₇:Eu²⁺, Dy³⁺ nanoparticles exhibit similarly spherical-like morphology with an average diameter of 29.9±0.36 nm in the TEM image (FIG. 19) and their XRD pattern (FIG. 20) can be indexed to a pure tetragonal Sr₂MgSi₂O₇ (JCPDS:75-1736). The Sr₂MgSi₂O₇:Eu²⁺, Dy³⁺ nanoparticles show a 123-fold increase in mechanoluminescence compared to ZnS:Ag, Co/ZnS particles at the same concentration (FIG. 21).

### DISCUSSION

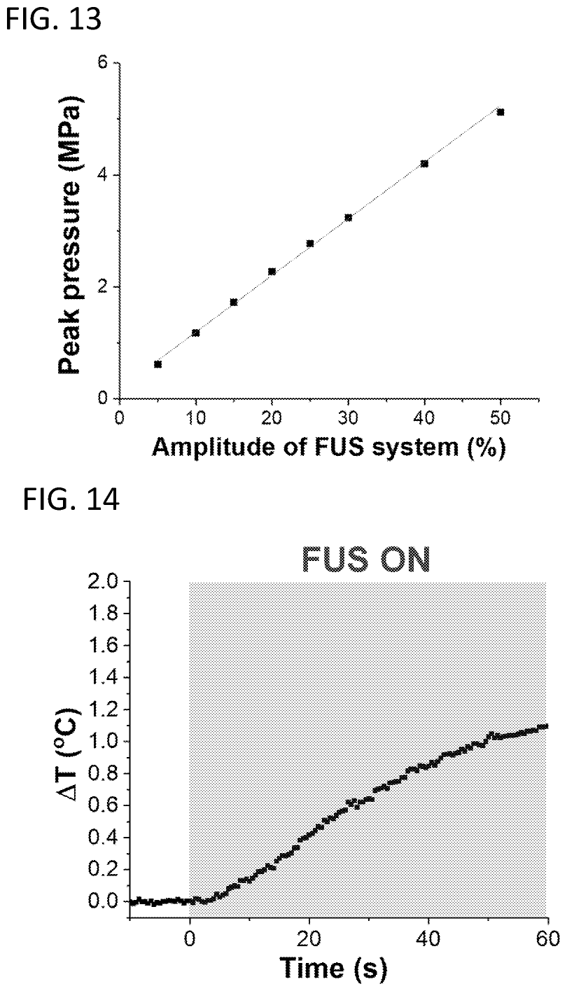

The results of sono-optogenetic stimulation have several fundamental differences from previous reports of non-specific neural activation with FUS. First, non-specific ultrasound neuromodulation usually exhibits improved efficacy with low frequencies below 1 MHz, while it has been reported to become increasingly difficult to demonstrate efficacious neuromodulation using ultrasound frequencies above 1 MHz (44, 47). This relationship between ultrasound frequency and neuromodulation efficacy imposes a challenge to spatially confine the FUS in the brain due to the inverse dependence of ultrasound wavelength, which determines the spatial resolution, on frequency. The method, in contrast, demonstrates efficacious neural stimulation with a center ultrasound frequency of 1.5 MHz, which results in a small in-plane focus of 0.7 mm×0.7 mm in the x and y dimensions. Second, a power density of 40 W/cm² was reported for 1.4 MHz ultrasound to achieve a success rate of 50% for muscle contraction in wild-type mice (44). In the experiments, a power density of merely 10 W/cm² was sufficient to activate circulating nanoparticles in the blood and produce enough photon flux to stimulate ChR2 neurons with visible twitches of the hindlimb in a reproducible manner. It was reason that the successful sono-optogenetic stimulation under such a low ultrasound power density was owing to the enhancement of mechanoluminescence by coating the ZnS:Ag, Co particles with an additional layer of ZnS, as the uncoated ZnS:Ag, Co nanoparticles were unable to elicit any limb motion under the same protocol (FIG. 16). Third, unlike non-specific neuromodulation with FUS that usually evokes bilateral hindlimb motion (44), the method demonstrates clear contralateral limb activation, owing to the specific expression of ChR2 in Thy1 neurons and spatially confined FUS to afford regional selectivity in the brain. Therefore, sono-optogenetics provides a minimally invasive and cell-type specific neuromodulation method with high spatial resolution and low power requirement.

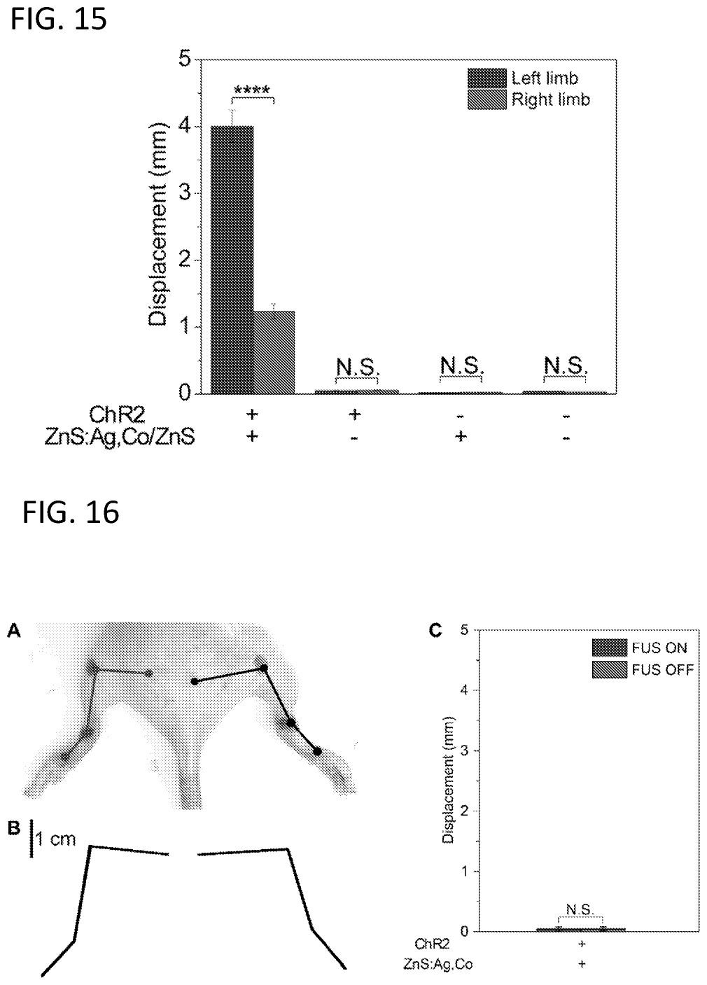

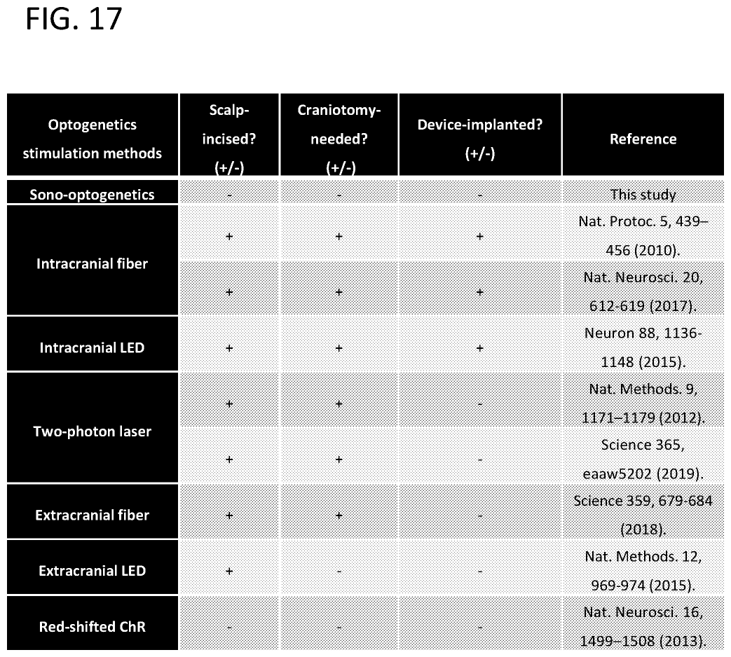

Compared to the existing optogenetic methods, sono-optogenetics represents the least invasive technique to implement optogenetic neuromodulation (FIG. 17). The most common protocol for in vivo optogenetic stimulation involves implantation of a fiber cannula (11, 48) or an LED (49) to the targeted brain region, imposing acute damage to the local neural tissue and chronic gliosis at the fiber interface (50, 51). To mitigate the invasiveness of implantation into the brain, recent advances in implementation of optogenetics take advantage of deeper tissue penetration of longer-wavelength photons by designing red-shifted opsins (7, 18), replacing conventional light sources with upconversion nanoparticles (21), and activating opsins via a two-photon process (7, 19). Despite these advances, scalp removal and craniotomy are usually required to meet the power requirement and spatial selectivity for optogenetic stimulation in the brain. The approach represents the first example of optogenetic neuromodulation in the brain of live animals without any invasive procedure to the scalp and skull, owing to the deep brain penetration of ultrasound, and the unique delivery method of light source via the intrinsic blood vasculature. In the approach, the circulating mechanoluminescent nanoparticles are not needed to physically cross the blood-brain barrier for optogenetic stimulation of neurons, owing to the sufficient penetration depth of 200 μm for 473-nm photons (52), and the pervasive cerebral vasculature penetrating into every region of the brain (53). In comparison to organic mechanophores that emit light during the irreversible break of chemical bonds (28), the rechargeability of ZnS:Ag, Co/ZnS nanoparticles with 400-nm excitation allows for repetitive optogenetic stimulation in the brain after a single intravenous injection, making the method suitable for animal studies that last hours to days. It has also demonstrated the lack of any noticeable tissue damage or pathological lesion in organs of mice injected with ZnS:Ag, Co/ZnS nanoparticles (FIG. 18), suggesting good biocompatibility of the circulation-delivered light sources for sono-optogenetic stimulation.

In summary, it has been achieved minimally invasive in vivo optogenetic stimulation in live mouse brain using a new sono-optogenetic method. Unlike conventional approaches of light delivery via a brain implant for optogenetic neuromodulation, sono-optogenetics takes advantage of the intrinsic circulatory system to deliver nanosized light sources, the ZnS:Ag, Co/ZnS nanoparticles, and makes use of the brain-penetrant ultrasound to rapidly switch these circulating nanoparticles on and off in specific brain regions. Sono-optogenetics demonstrates efficacious ChR2 activation in vitro and neuromodulation with motor behavioral changes in vivo, the latter of which can be accomplished through intact scalp and skull to minimize any damage to the brain tissue. Engineering of the trap states by varying the dopant ions and dopant concentrations in the nanoparticle matrix could lead to more efficient sono-optogenetic activation of different opsins with less ultrasound power density (35, 54). It was envisage that sono-optogenetics provides a unique tool of rapid screening of different target regions in the brain for optogenetic neural modulation, owing to the ease of changing the location of ultrasound focus in the brain by eliminating fiber optic implantation. Furthermore, sono-optogenetics can be used in other regions of the central and peripheral nervous systems, as well as in other organs such as the heart and lungs, which are usually refractory to fiber implantation due to structural and functional constraints, for precise modulation with optogenetic control of cell activity. In addition, reduction of the footprint and the weight of the ultrasound transducer, as well as the use of ultraflexible neural probes with neural-tissue-like mechanical compliance (55), may allow sono-optogenetic stimulation of deep-brain regions with simultaneous electrophysiology in a behavioral setting. It was envision that this approach could also be extended to applications in much deeper brain regions in larger animals owing to the penetration depth of ultrasound reaching several centimeters.

### Materials and Methods

### Synthesis of Mechanoluminescent Nanoparticles.

Chemicals were purchased from Sigma-Aldrich unless otherwise claimed.

Synthesis of ZnS:Ag, Co nanoparticles. Zinc acetate (383317, 184 mg, 1 mmol) was weighed and transferred to a 100-mL round-bottom flask followed by dissolution in deionized (DI) water (40 mL) at room temperature by a magnetic stirring hotplate (Cimarec+ Stirring Hotplate, Thermo Fisher Scientific, Inc., Waltham, Mass.). AgNO₃ (209139, 2.3 mM in DI water, 1.2 mL) and cobalt(II) acetate (399973, 0.8 mM in DI water, 25 μL) were added into the zinc acetate solution and then the solution was stirred at room temperature for 5 min. Complete dissolution of cobalt(II) acetate before transferal to the zinc acetate solution is critical to successful synthesis of ZnS:Ag, Co mechanoluminescent materials, since cobalt(II) acetate is hygroscopic and can hydrolyze into insoluble cobalt(II) hydroxide. 3-Mercaptopropionic acid (M5801, 0.4 mL) was then added into the solution under stirring and the solution became turbid. NaOH (795429, 2 M in DI water) was added into the mixture to adjust the pH to 10.5 and the solution became clear. After that, Na₂S.9H₂O (S2006, 0.46 M in DI water, 1 mL) was added into solution under stirring. The mixture was transferred into a 50-mL Teflon-lined stainless steel autoclave for hydrothermal reaction in an oven (Heratherm OMH60 Lab Oven, Thermo Fisher Scientific, Inc., Waltham, Mass.) at 120° C. for 24 h to yield a white colloid (product 1) (56). The ZnS:Ag, Co nanoparticles were then purified by addition of 20 mL absolute ethanol followed by centrifugation (8000 rpm, 8 min). The supernatant was decanted and the nanoparticles were purified further by re-dispersion into 10 mL DI water by sonication, precipitation by 5 mL ethanol, and then centrifugation (Thermo Scientific Sorvall Legend X1R Centrifuge, Thermo Fisher Scientific, Inc., Waltham, Mass.) at 8000 rpm for 10 min. This procedure was repeated twice. The precipitates were dried by lyophilization and transferred into a 5-mL porcelain crucible for calcination in a tube furnace at 800° C. for 3 h under argon atmosphere. The obtained materials were then dispersed in a mixed solvent containing 20 mL absolute ethanol and 10 mL CH₂Cl₂ in a 50-mL centrifuge tube and sonicated for 1 h. After that, the dispersion was centrifuged at 1000 rpm for 10 min. The supernatant was collected and centrifuged at 8000 rpm for 10 min to collect the precipitates. The precipitates were then re-dispersed into 30 mL ethanol under sonication and then collected by centrifugation at 8000 rpm for 10 min. This step was repeated 3 times. The resulting ZnS:Ag, Co nanoparticles were dried by lyophilization to yield a colorless powder (yield: 86.5 mg, 88.1%).

Synthesis of ZnS:Ag, Co/ZnS nanoparticles. Into a 100-mL round-bottom flask with magnetic stir bar was added 1, zinc acetate (0.368 g), and Na₂S.9H₂O (0.46 M in DI water, 2 mL). After stirring for 5 minutes at room temperature, the solution was transferred into a 50-mL Teflon-lined stainless steel autoclave for hydrothermal reaction in an oven (Heratherm OMH60 Lab Oven, Thermo Fisher Scientific, Inc., Waltham, Mass.) at 120° C. for 24 h to yield a white colloid of ZnS:Ag, Co/ZnS nanoparticles. Purification and calcination procedures were the same as for ZnS:Ag, Co nanoparticles (yield: 251.5 mg, 85.8%).

Synthesis of undoped ZnS nanoparticles without sonoluminescent properties. Zinc acetate (383317, 184 mg, 1 mmol) was weighed and transferred to a 100-mL round-bottom flask followed by dissolution in deionized (DI) water (40 mL) at room temperature by a magnetic stirring hotplate (Cimarec+ Stirring Hotplate, Thermo Fisher Scientific, Inc., Waltham, Mass.). 3-Mercaptopropionic acid (M5801, 0.4 mL) was then added into the solution under stirring and the solution became turbid. NaOH (795429, 2 M in DI water) was added into the mixture to adjust the pH to 10.5 and the solution became clear. After that, Na₂S.9H₂O (S2006, 0.46 M in DI water, 1 mL) was added into solution under stirring. The mixture was transferred into a 50 mL Teflon-lined stainless steel autoclave for hydrothermal reaction in an oven (Heratherm OMH60 Lab Oven, Thermo Fisher Scientific, Inc., Waltham, Mass.) at 120° C. for 24 h to yield a white colloid of ZnS nanoparticles. Purification and calcination procedures were the same as for ZnS:Ag, Co nanoparticles (yield: 84.7 mg, 86.9%).

### Surface Modification of ZnS:Ag, Co/ZnS Nanoparticles.

100 mg of ZnS:Ag, Co/ZnS nanoparticles and 100 mg of 1,2-distearoyl-sn-glycero-3-phosphoethanolamine-N-[methoxy(polyethylene glycol)-2000] (DSPE-mPEG, Avanti Polar Lipids, Alabaster, Ala.) were added into a 100 mL round bottom flask followed by the addition of 10 mL dichloromethane (Sigma, 270997, St. Louis, Mo.). The mixture was sonicated for 1 min and then a rotovap was used to remove dichloromethane in the mixture. After that, 20 mL of DI water was added into the dried mixture and DSPE-mPEG modified nanoparticles were dispersed in water by sonication. The dispersion was then centrifuged at 8000 rpm for 10 min and the precipitates (DSPE-mPEG modified nanoparticles) were collected. 20 mL DI water was added into the precipitates and the mixture was sonicated to disperse the nanoparticles.

### Transmission Electron Microscopy (TEM) Imaging of ZnS:Ag, Co/ZnS Nanoparticles.

Three drops (2.5 μL/drop) of ZnS:Ag, Co/ZnS nanoparticle suspension (200 μg mL⁻¹) was deposited on a formvar/carbon film coated copper grid (Ted Pella, Inc., Redding, Calif.) and dried in a desiccator for at least 2 h. Afterwards, TEM images of ZnS:Ag, Co/ZnS nanoparticles were captured on an FEI Tecnai Transmission Electron Microscope (Field Electron and Ion Company, FEI, Hillsboro, Oreg.).

### Dynamic Light Scattering (DLS) of ZnS:Ag, Co/ZnS Nanoparticles.

Aliquot of the abovementioned ZnS:Ag, Co/ZnS suspension was diluted with 1× phosphate buffered saline (PBS, pH 7.4) and Dulbecco's Modified Eagle Medium (DMEM) supplemented with 10% fetal bovine serum (FBS) to a final concentration of 200 μg mL⁻¹. Then, hydrodynamic diameters of diluted ZnS:Ag, Co/ZnS nanoparticles in these two solutions were measured by DLS on a Malvern Nano-ZS Particle Sizer (Malvern Panalytical Ltd, Malvern, UK).

### UV-Vis-NIR Absorption Spectroscopy of ZnS:Ag, Co/ZnS Nanoparticles.

UV-Vis-NIR absorption spectrum of ZnS:Ag, Co/ZnS nanoparticle suspension was measured by a Cary 6000i spectrophotometer (Agilent, Santa Clara, Calif.) with a total path length of 1 mm, background-corrected for contribution from water and the cuvette. The measured range was 200-800 nm.

### Photoluminescence Spectroscopy of ZnS:Ag, Co/ZnS Nanoparticles.

Photoluminescence spectrum of ZnS:Ag, Co/ZnS nanoparticle suspension was measured by a Horiba FluoroLog Fluorimeter spectrophotometer (HORIBA Scientific, Piscataway, N.J.) in a quartz cuvette. The excitation wavelength was 365 nm and the measured range of photoluminescence was 400-650 nm.

### Mechanoluminescence Spectroscopy of ZnS:Ag, Co/ZnS Nanoparticles.

ZnS:Ag, Co/ZnS nanoparticles were mixed with polydimethylsiloxane (PDMS) to form a flat, cylindrical phantom (1.6 cm diameter×0.2 cm thickness) with the nanoparticle concentration of 75 mg mL⁻¹. The nanoparticles-containing PDMS sample was clamped and fixed by a custom holder with alligators and placed on top of a focused ultrasound (FUS) transducer coupled with degassed water bag (Image Guided Therapy, Pessac, France) at room temperature, such that the FUS was focused inside and near the upper surface of the phantom. The center frequency of the transducer was 1.5 MHz, and the peak pressure at the focus was 1.86 MPa. A pulse train of 100 ms duration was delivered with a repetition frequency of 1 Hz. During the FUS application, a fiber-coupled spectrometer (OCEAN-HDX-VIS-NIR, Ocean Optics, Largo, Fla.) was used to collect the emitted mechanoluminescence by placing the end of the optical fiber on the upper surface of the phantom opposite the FUS transducer. The spectral range of measurement was 400-650 nm, with a wavelength resolution of 0.366 nm and an acquisition time of 4 s.

### Artificial Circulation System to Mimic Blood Circulation.

1.5 mm inner diameter (I.D.) and 3.0 mm outer diameter (O.D.) Tygon® tubing with a total length of ca. 35 cm was used for making the artificial circulation system to mimic blood circulation in live animals. The tubing was connected to a cylindrical piece of PDMS (2 cm length×1.2 cm diameter) with a tunnel (diameter=2 mm) to complete the circulation (see FIG. 9). The tubing was filled with a PBS suspension of ZnS:Ag, Co/ZnS nanoparticles at a concentration of 8 mg mL⁻¹ to mimic the concentration circulating in the blood stream. A peristaltic pump (Model 720, Harvard Apparatus, Cambridge, Mass.) was used to circulate the solution inside the tubing at a rate of 4.75 m/min. To avoid air bubbles in the circulation system, one end of the tubing was connected to the PDMS tunnel first and the pipeline was filled with 200 μL of ZnS:Ag, Co/ZnS nanoparticle dispersion through one end of the PDMS (liquid inlet), before the other end of tubing (liquid outlet) was connected to the PDMS tunnel when the pump was running. FUS was applied from the aforementioned transducer at room temperature and delivered to the tunnel inside the PDMS phantom to mimic a deep vessel embedded in the brain tissue. FUS was applied with a repetition frequency of 1 Hz and a duty cycle of 2% for latency time measurement (FUS on: 20 ms; FUS off: 980 ms). All other parameters of FUS are the same as mechanoluminescence spectroscopy measurement. A 400-nm excitation light was provided from a light-emitting diode (LED; Mouser Electronics, Mansfield, Tex.) at a power density of 10.2 mW/mm² to recharge the ZnS:Ag, Co/ZnS nanoparticles while they were circulating in the artificial circulation system. A fiber-coupled spectrometer (OCEAN-HDX-VIS-NIR, Ocean Optics, Largo, Fla.) was used to collect the emitted mechanoluminescence at 470 nm with a data acquisition rate of 20 Hz, by inserting a fiber optic cannula with a 400-μm core (Thorlabs, Newton, N.J.), which was connected to the end of the optical fiber, into the piece of PDMS until the end of cannula was 0.25 mm away from the tunnel.

### Viral Vector Construction.

Viral vectors used in this work include: pCMV-hChR2(H134R)-mCherry plasmid was constructed by Vector Biolabs (Malvern, Pa.).

### Cell Culture and Transfection.

NaV 1.3 KIR 2.1 human embryonic kidney (HEK) cells with overexpressed voltage-gated sodium channels (NaV) and inwardly rectifying potassium channels (KIR) were purchased from ATCC (Manassas, Va.). Cell culture was maintained in DMEM medium supplemented with 10% FBS. NaV 1.3 KIR 2.1 HEK cells were transfected with 7.5 μL of Lipofectamine® 3000 (Invitrogen, Carlsbad, Calif.) with 2500 ng of total DNA of pCMV-hChR2(H134R)-mCherry plasmids (Vector Biolabs, Malvern, Pa.) in Opti-MEM medium (Gibco), and used for in vitro sono-optogenetic stimulation 3-5 days after transfection.

In Vitro Sono-Optogenetic Stimulation with the Artificial Circulation System.

NaV 1.3 KIR 2.1 HEK cells transfected with ChR2 or untransfected NaV 1.3 KIR 2.1 HEK cells were plated in a microelectrode array (MEA, MEA2100-System, Multi Channel Systems MCS GmbH, Reutlingen, Germany). Two days after plating the cells, the MEA was inspected under an inverted infinity and phase contrast microscope (Fisher Scientific, Hampton, N.H.) to ensure a confluency between 60% and 80% and sufficient coverage of the electrodes with adherent HEK cells. Then the MEA was placed in a headstage (MEA2100-HS, Multi Channel Systems MCS GmbH, Reutlingen, Germany), which recorded extracellular action potentials from HEK cells during sono-optogenetic stimulation.

An artificial circulation system was assembled with an 8-mm portion of the polyethylene tubing placed between the MEA and the FUS transducer coupled with a degassed water bag. ZnS:Ag, Co/ZnS nanoparticles suspended in PBS solution at a concentration of 8 mg mL⁻¹ were loaded into the tubing and allowed to circulate by the peristaltic pump mentioned above. The 400-nm excitation light was confined to the distal end of the circulatory tubing with a power density of 10.2 mW mm⁻² and covered with black-tape-coated aluminum foil to minimize light leakage to the MEA that would otherwise stimulate ChR2-expressing NaV 1.3 KIR 2.1 HEK cells optically. The distance between the MEA and the FUS transducer was adjusted such that the FUS focus was located near the inner surface of the MEA chamber which the HEK cells adhered to. FUS was applied with a repetition frequency of 1 Hz and a duty cycle of 10% (i.e., FUS on: 100 ms; FUS off: 900 ms), while the MEA headstage measured the extracellular single-unit neuron activity simultaneously. One of the built-in channels in the MEA was routed to connect to analog output of the function generator that drives the FUS transducer, enabling precise recording of the timestamp when each FUS pulse was turned on. For control experiments in which no ZnS:Ag, Co/ZnS nanoparticles were used as the medium for sono-optogenetic stimulation, PBS solution was used in the artificial circulation system instead (FIG. 3).

### Data Analysis of In Vitro Electrophysiology.

The electrophysiological recording data was analyzed offline. In brief, raw recording data was loaded in a user-written MATLAB program that performs thresholding to extract single-unit spikes. The threshold was set at 50 μV based on estimation of peak amplitudes of measured extracellular action potentials. A 3-ms interval (1 ms before the main peak, and 2 ms after the main peak) was used to include the entire waveform of each single-unit spike, and all spikes were overlaid to demonstrate reproducible firing triggered by sono-optogenetic stimulation. The latency time of sono-optogenetic stimulation was calculated as the difference between the timestamp when each FUS pulse was delivered, which was recorded in one of the MEA channels connected to analog output of the FUS functional generator, and the peak location of the succeeding action potential, which was recorded by a separate MEA channel in close proximity to the firing cell.

### Vertebrate Animal Subjects.

Adult (20-30 g) male C57BL/6J mice (4 weeks old, Jackson Laboratory, Bar Harbor, Me.) and Thy1-ChR2-YFP mice (4 weeks old, Jackson Laboratory, Bar Harbor, Me.) were the vertebrate animal subjects used in this study. All procedures performed on the mice were approved by Stanford University's Administrative Panel on Laboratory Animal Care (APLAC). The animal care and use programs at Stanford University meet the requirements of all federal and state regulations governing the humane care and use of laboratory animals, including the USDA Animal Welfare Act, and PHS Policy on Humane Care and Use of Laboratory Animals. The laboratory animal care program at Stanford is accredited by the Association for the Assessment and Accreditation of Laboratory Animal Care (AAALAC International). Animals were group-housed on a 12 h: 12 h light: dark cycle in the Stanford University's Veterinary Service Center (VSC) and fed with food and water ad libitum as appropriate.

In Vivo Sono-Optogenetic Stimulation with Circulation-Delivered ZnS:Ag, Co/ZnS Nanoparticles.

ZnS:Ag, Co/ZnS nanoparticles were delivered into blood circulation via tail-vein injection. Mice were anesthetized by intraperitoneal injection of a mixture of 16 mg/kg ketamine (KetaVed®, Vedco, Inc., St. Joseph, Mo.) and 0.2 mg/kg dexdomitor (Dexmedesed™, Dechra Veterinary Products, Overland Park, Kans.). The degree of anesthesia was verified via the toe pinch method before the procedure started. To maintain the body temperature and prevent hypothermia of the surgical subject, a homeothermic blanket (Harvard Apparatus, Holliston, Mass.) was set to 37° C. and placed underneath the anesthetized mouse (World Precision Instruments, Inc., Sarasota, Fla.). Vet ointment (Puralube®, Dechra Veterinary Products, Overland Park, Kans.) was applied on both eyes of the mouse to moisturize the eye surface throughout the experiment. Hair removal lotion (Nair®, Church & Dwight, Ewing, N.J.) was used for depilation of the mouse head, back and both hindlimbs. The hair over the mouse head was removed to help form a continuous interface between the scalp and the water bag to reduce reflection of applied ultrasound at the air/skin interface. The hair over the mouse back and hindlimbs was removed to allow for marking the joints of both hindlimbs with different colors (FIG. 4) and tracking the limb trajectories during sono-optogenetic stimulation later. ZnS:Ag, Co/ZnS nanoparticles of which the surface were modified with 1,2-distearoyl-sn-glycero-3-phosphoethanolamine-N-[methoxy(polyethylene glycol)-2000] (ammonium salt) (Avanti Polar Lipids, Alabaster, Ala.), dispersed in PBS with a concentration of 80 mg/mL (200 μL) were injected into mouse through tail vein by insulin syringes with 30G needle gauge.

After intravenous injection of ZnS:Ag, Co/ZnS nanoparticles, the mouse was positioned in the built-in stereotaxic frame of the FUS system. The mouse head was fixed by ear bars which are equipped in the animal bed of the FUS system. FUS transducer (1.5 MHz) integrated with a customized water bag manufactured by Image Guided Therapy was placed on mouse head with intact scalp. The stereotaxic coordinates of the secondary motor cortex (M2) are: anteroposterior (AP)+1.0 mm, mediolateral (ML)+0.5 mm, dorsoventral (DV)−0.5 mm (57). The height of the water bag was tuned by adjusting the volume of water with a syringe connected to the degassing system. The 400-nm LED (10.2 mW/mm²; Mouser Electronics, Mansfield, Tex.) was positioned near the jugular vessels in the neck of the mouse, with extra caution to ensure no direct illumination of the brain. FUS was applied with a repetition frequency of 1 Hz and a duty cycle of 10% (i.e., FUS on: 100 ms; FUS off: 900 ms), while a video camera was used to capture the motions of the mouse's both hindlimbs during sono-optogenetic stimulation. The hindlimb joint locations, which were marked by different-colored dots, were extracted by a user-written MATLAB program for plotting the hindlimb kinematics. Limb displacement was analyzed by computing the maximum displacement of the toe marker (

### Estimation of Ultrasound Power Density at the Focus in Brain Tissue.

Standard procedures were followed to measure the spatial peak pulsed average intensity (I_{SPPA}) at the ultrasound focus in the brain tissue (44). Specifically, a hydrophone was used to measure the pressure at the focus of the ultrasound transducer in water. I_{SPPA} in the brain tissue was calculated as follows,

$${I}_{SPPA} = \frac{a}{T} {\int }_{0}^{T} \frac{{P}^{2}}{ρ v} dt$$

where a is the attenuation of focused ultrasound due to the roof of the mouse skull and is measured as −2 dB (0.63) at 1.5 MHz (44), T is the period of a complete pressure waveform applied by the FUS transducer, P is the pressure at a particular output amplitude determined by the calibration curve in FIG. 13, ρ is the density of brain tissue (1040 kg/m³), and v is the speed of sound in the brain tissue (1560 m/s). I_{SPPA} is calculated as 10.0 W/cm² at an output amplitude of 20% and frequency of 1.5 MHz, which are the parameters used in the in vivo experiments.

### Measurement of Luminescence Intensity Triggered by FUS in Brain Tissue.

The intensity of 470-nm luminescence produced by circulating ZnS:Ag, Co/ZnS nanoparticles was measured in cerebral vessels as they were activated by brain-penetrant FUS by inserting a fiber optic cannula (CFMXD10, Thorlabs, Newton, N.J.) coupled to an optical fiber (M125L01, Thorlabs, Newton, N.J.), which was then connected to a photomultiplier tube (PMT) for power measurement (PMT1001, Thorlabs, Newton, N.J.), into the FUS focus. Unlike conventional light intensity measurement for fiber-coupled optogenetic stimulation, in which the optical fiber outfitted by a LED or a laser module can be directly connected to the power meter for measurement outside the brain before implantation, quantification of light emission power from circulating ZnS:Ag, Co/ZnS nanoparticles could not be performed outside the brain tissue and had to be carried out in situ. However, it was difficult, if not impossible, to accurately measure the light intensity received by neurons in the immediate vicinity of FUS-activated blood vessels, and any measurement by directly placing a power-measuring optical fiber into the brain during FUS application would underestimate the actual power density for the following two reasons. First, the intensity of blue light rapidly decays in the brain tissue as a result of endogenous light absorbers and scatterers (56). Second, the optical fiber used for power measurement usually has a small numerical aperture to meet total internal reflection criterion, leading to inefficient light collection and coupling into the core of the fiber (7).

Therefore, to mitigate these challenges, two fiber optic cannulas (CFMXD10, Thorlabs, Newton, N.J.) were inserted, one of which (‘the calibration cannula’, M125L01, Thorlabs, Newton, N.J.) was used to deliver 470-nm light from a blue LED (M470F3, Thorlabs, Newton, N.J.) with known power output at the tip of the cannula, and the other of which (‘the measuring cannula’, M125L01, Thorlabs, Newton, N.J.) was used to collect locally emitted blue photons in the brain tissue, both into the same M2 region of the brain (stereotaxic coordinates: AP+1.0 mm, ML+0.5 mm, DV+0.5 mm). The distance between the ends of the two optical fibers was 0.5 mm. Besides, ZnS:Ag, Co/ZnS nanoparticles were delivered into blood circulation at the same dose as described in ‘In vivo sono-optogenetic stimulation with circulation-delivered ZnS:Ag, Co/ZnS nanoparticles’ above, charged by the 400-nm excitation light source, and activated by FUS to produce local luminescence. When the ‘calibration cannula’ delivered light and the FUS was off, the output power of the LED was adjusted such that the power measured by the ‘measuring cannula’ was the same as when FUS was on to trigger emission from ZnS:Ag, Co/ZnS nanoparticles and the ‘calibration cannula’ delivered no light. Then the power output from the ‘calibration cannula’ with the matching settings was used to estimate the equivalent power produced by ZnS:Ag, Co/ZnS nanoparticles triggered by FUS.

### Blood Circulation Study

An amount of 20 μL of blood was collected from tail vein at various time points (7.5 min, 30 min, 1 h, 2 h, 4 h, 8 h and 24 h) after injection of 200 μL of 80 mg/mL ZnS:Ag, Co/ZnS nanoparticles into the mouse tail vein (n=3). Each blood sample was diluted 1000× and added to a quartz cuvette. Photoluminescence spectrum of the diluted blood sample was measured by a Horiba FluoroLog Fluorimeter spectrophotometer (HORIBA Scientific, Piscataway, N.J.). The peak fluorescence intensity was taken from each spectrum, and compared with that of a ZnS:Ag, Co/ZnS nanoparticle solution with known concentrations for determination of the ZnS:Ag, Co/ZnS concentration in the blood samples. A first order exponential decay was fitted to the data to extract the circulation half-life of intravenously injected nanoparticles.

### Histological Study

On day 3 and day 28 after intravenous administration of ZnS:Ag, Co/ZnS nanoparticles, injected mice were euthanized and their major organs (brain, heart, lung, liver, spleen and kidney) were harvested and fixed in 4% paraformaldehyde. After 48 h of tissue fixation, these organs were embedded in paraffin and sectioned to 10-μm slices. Later, the slices were stained with hematoxylin and eosin (H&E), followed by imaging under an inverted infinity and phase contrast microscope (Fisher Scientific, Hampton, N.H.).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of contacting a tissue of a subject with light, comprising:
applying an ultrasound signal to a photoexcited mechanoluminescent particle while the mechanoluminescent particle is in proximity to the tissue of the subject, thereby causing the mechanoluminescent particle to emit light that contacts the tissue.

2. The method of claim 1, wherein the tissue comprises a neuron that comprises a photosensitive protein, and wherein the emitted light modulates the photosensitive protein.

3. The method of claim 2, wherein the modulation results in hyperpolarization of the neuron.

4. The method of claim 2, wherein the modulation results in depolarization of the neuron.

5. The method of any one of claims 2-4, further comprising genetically modifying the neuron to express the photosensitive protein.

6. The method of any one of claims 2-5, wherein the photosensitive protein is a channelrhodopsin-2 (ChR2), a Volvox carteri light-activated protein (VChR1), a iC++, a ChRmine, or a halorhodopsin (HPHR).

7. The method of claim 6, wherein the photosensitive protein is a halorhodopsin.

8. The method of claim 7, wherein the halorhodopsin is a halorhodopsin from Natronomonas (NpHR).

9. The method of claim 1, wherein the tissue comprises a group of compounds that causes genetic modification to the tissue after absorbing the emitted light.

10. The method of claim 9, wherein the group of compounds comprises a CRISPR compound and a Cas9 compound.

11. The method of claim 1, further comprising fluorescently imaging the tissue by measuring the light emitted by the mechanoluminescent particle.

12. The method of claim 11, wherein the measuring comprises measuring light with a light measurement device inserted into the gastrointestinal tract of the subject.

13. The method of claim 1, wherein the tissue comprises a photosensitizer that is contacted by the emitted light, thereby generating a reactive oxygen species.

14. The method of claim 13, wherein the subject has a disease and the method is a method of treating the subject for the disease by generating a reactive oxygen species in the tissue.

15. The method of claim 14, wherein the disease is an infection by a microorganism.

16. The method of claim 14, wherein the disease is cancer and the tissue comprises cancer cells.

17. The method of any one of claim 1-16, further comprising administering the mechanoluminescent particle to the subject.

18. The method of claim 17, wherein the administering comprises intravenously injecting a liquid that comprises the mechanoluminescent particle into the subject.

19. The method of any one of claims 1-18, further comprising photoexciting the mechanoluminescent particle before the application of the ultrasound signal.

20. The method of claim 19, wherein the photoexciting comprises applying excitation light to the mechanoluminescent particle while the particle is outside the subject.

21. The method of claim 19, wherein the photoexciting comprises applying excitation light to an external surface of the subject after administering the mechanoluminescent particle to the subject such that the excitation light photoexcites the mechanoluminescent particle.

22. The method of claim 21, wherein the external surface is skin covering a blood vessel and the mechanoluminescent particle is in the blood vessel during the application of light.

23. The method of claim 22, wherein the blood vessel is 20 mm or less below the skin.

24. The method of any one of claims 21-23, wherein the external surface of the subject is part of the head or the neck of the subject.

25. The method of any one of claims 22-24, wherein the blood vessel is a facial artery or a jugular vein.

26. The method of any one of claims 19-25, further comprising administering the mechanoluminescent particle to the subject, wherein the time between the administering and the photoexciting is 60 minutes or less.

27. The method of any one of claims 20-26, wherein 80% or more of the photons of the excitation light have a wavelength ranging from 350 nm to 450 nm.

28. The method of any one of claims 1-27, wherein 80% or more of the photons of the light emitted by the mechanoluminescent particle have a wavelength ranging from 400 nm to 1700 nm.

29. The method of any one of claims 1-28, wherein the mechanoluminescent particle is a nanoparticle having a dimension ranging from 1 nm to 1 μm.

30. The method of claim 29, wherein the dimension ranges from 30 nm to 250 nm.

31. The method of any one of claims 1-30, wherein the mechanoluminescent particle is a sphere.

32. The method of any one of claims 1-31, wherein the mechanoluminescent particle comprises an inorganic material.

33. The method of claim 32, wherein the mechanoluminescent particle comprises a semiconductor material, an insulator material, or both, wherein at least one of the semiconductor material and the insulator material has a bandgap energy ranging from 2 eV to 5 eV.

34. The method of any one of claims 1-33, wherein the mechanoluminescent particle comprises zinc sulfide (ZnS); tridymite (X¹Al₂O₄ wherein X¹ is Sr, Ca, Ba, or a combination thereof); melilite (X²₂ESi₂O₇ wherein X² is Ca, Sr, Ba, or a combination thereof, E is Mg); SrMg₂(PO₄)₂; perovskite (Ba₁₋ₓCaₓTiO₃ wherein 0.25<x<0.8); BaSi₂O₂N₂; SrSi₂O₂N₂; CaZr(PO₄)₂; or a combination thereof.

35. The method of claim 34, wherein the mechanoluminescent particle comprises zinc sulfide (ZnS).

36. The method of claim 35, the zinc sulfide is wurtzite zinc sulfide.

37. The method of any one of claims 1-36, wherein the mechanoluminescent particle is a core-shell particle comprising an un-doped shell and a core doped with a first and second dopants.

38. The method of claim 37, wherein the first dopant is Co²⁺ and the second dopant is Ag⁺.

39. The method of claim 38, wherein the amount of Co²⁺ is 0.001 mol % to 1 mol %.

40. The method of claim 38 or 39, wherein the amount of Ag⁺ is 0.001 mol % to 1 mol %.

41. The method of claim 34, wherein the mechanoluminescent particle comprises melilite (X²₂ESi₂O₇ wherein X² is Ca, Sr, Ba, or a combination thereof, E is Mg).

42. The method of claim 41, wherein the melilite is doped with Eu²⁺, Dy³⁺, or a combination thereof.

43. The method of any one of claims 1-42, wherein the mechanoluminescent particle comprises an organic material.

44. The method of claim 43, wherein the organic material comprises a N-(4-trifluoromethylphenyl)phthalimide group.

45. The method of any one of claims 1-44, wherein the mechanoluminescent particle comprises a biocompatible coating.

46. The method of claim 45, wherein the biocompatible coating comprises polyethylene glycol or a derivative thereof.

47. The method of any one of claims 1-46, wherein the ultrasound signal is a focused ultrasound signal (FUS).

48. The method of any one of claims 1-47, wherein the ultrasound signal has a frequency ranging from 150 kHz to 15 MHz.

49. The method of any one of claims 1-48, wherein the ultrasound signal is repeated at a rate ranging from 0.2 repetitions per second to 5 repetitions per second.

50. The method of any one of claims 1-49, wherein the ultrasound signal has a spatial peak pulsed average intensity (I_{SPPA}) at a target neuron ranging from 1 W/cm² to 100 W/cm².

51. The method of claim 50, wherein the spatial peak pulsed average intensity at a target neuron ranges from 5 W/cm² to 15 W/cm².

52. The method of any one of claims 1-51, wherein the time interval between the application of the ultrasound signal and the emission of light from the photoexcited mechanoluminescent particle is 9 ms or less.

53. The method of any one of claims 1-52, wherein the time interval between the photoexcitation of the mechanoluminescent particles and the application of ultrasound ranges from 1 second to 60 minutes.

54. A system for contacting a tissue of a subject with light, the system comprising:
an ultrasound device configured to apply an ultrasound signal to a mechanoluminescent particle in proximity to the tissue, thereby causing the mechanoluminescent particle to emit light that contacts the tissue.

55. The system of claim 54, wherein the ultrasound signal is a focused ultrasound signal.

56. The system of any one of claims 54-55, further comprising the mechanoluminescent particle.

57. The system of any one of claims 54-56, further comprising an excitation light source that emits excitation light that can photoexcite the mechanoluminescent particle.

58. The system of any one of claims 54-57, further comprising an apparatus comprising a liquid a nucleic acid comprising a nucleotide sequence encoding for a photosensitive protein.

59. The system of any one of claims 54-57, further comprising a light measurement device configured to measure the light emitted from the mechanoluminescent particle.

60. The system of claim 59, wherein the light measurement device is configured to be inserted into the gastrointestinal tract of the subject.

61. The system of any one of claims 54-57, further comprising an apparatus comprising a liquid comprising a photosensitizer that can generate a reactive oxygen species by being contacted with the light emitted by the mechanoluminescent particle.

62. A kit comprising two or more of:
a mechanoluminescent particle;
a device for administering the mechanoluminescent particle to a subject;
a device for emitting light onto an external surface of a subject such that a mechanoluminescent particle in the subject is photoexcited; and
a device for emitting ultrasound.
